SEQUENCE LISTING (97 sequences)

SEQ ID NO: 1 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 2 - protein, 390 aa, Homo sapiens
TPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAP

SEQ ID NO: 3 - protein, 391 aa, Oryctolagus cuniculus
TPKIEDLPDHIEVNTGKFNPICKASGWPLPANEEMTLVKPDGTVLHPKDFNHTENFSVAIFTIHRILPLDSGVWVCSVNTVAGMVEKPFNISVKVLPKPLNAPNVIDTGHNFAVINISSEPYFGDGPIKSKKLLYKPVNDYEAWRHIQVTNEIVTLNYLEPRTEYELCVQLIRRGEGGEGHPGPVRRFTTASIGLPPPQGLILLPKSQTTLNLTWQPIFPSSEDDFYVEVERRSVQIKSDQQNIKVPGNLTSVLLNNLHPREQYVVRARVNTKAQGEWSEDLTAWTLSDIVPPQPENIKISNITDSSAVISWTILDGYSISSIIIRYKVQGKNEDQHIDVKIKNATITQYQLKGLEPETAYQVDMFAENNIGSSNPAFSHELMTLPESQAP

SEQ ID NO: 4 - protein, 389 aa, Mus musculus
TPQIEDLPDHIEVNSGKFNPICKASGWPLPTSEEMTLVKPDGTVLQPNDFNYTDRFSVAIFTVNRVLPPDSGVWVCSVNTVAGMVEKPFNISVKVLPEPLHAPNVIDTGHNFAIINISSEPYFGDGPIKSKKLFYKPVNQAWKYIEVTNEIFTLNYLEPRTDYELCVQLARPGEGGEGHPGPVRRFTTASIGLPPPRGLSLLPKSQTALNLTWQPIFTNSEDEFYVEVERRSLQTTSDQQNIKVPGNLTSVLLSNLVPREQYTVRARVNTKAQGEWSEELRAWTLSDILPPQPENIKISNITDSTAMVSWTIVDGYSISSIIIRYKVQGKNEDQHIDVKIKNATVTQYQLKGLEPETTYHVDIFAENNIGSSNPAFSHELRTLPHSPAS

SEQ ID NO: 5 - protein, 12 aa, Homo sapiens
TLSDILPPQPEN

SEQ ID NO: 6 - protein, 14 aa, Homo sapiens
FAENNIGSSNPAFS

SEQ ID NO: 7 - protein, 5 aa, synthetic construct
SYWMN

SEQ ID NO: 8 - protein, 17 aa, synthetic construct
MIHPSDSETRLNQKFMD

SEQ ID NO: 9 - protein, 6 aa, synthetic construct
GLYGNS

SEQ ID NO: 10 - protein, 11 aa, synthetic construct
RASQDIGISLN

SEQ ID NO: 11 - protein, 7 aa, synthetic construct
ATSSLDS

SEQ ID NO: 12 - protein, 9 aa, synthetic construct
LQYASSPYT

SEQ ID NO: 13 - protein, 115 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSS

SEQ ID NO: 14 - protein, 106 aa, synthetic construct
DIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEI

SEQ ID NO: 15 - protein, 242 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSGGGGSGGGGSGGGGSGGGGSDIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEIK

SEQ ID NO: 16 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 445 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 441 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 108 aa, synthetic construct
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 10 aa, synthetic construct
GGGGSGGGGS

SEQ ID NO: 22 - protein, 20 aa, synthetic construct
GGGGSGGGGSEPKSSDKTHT

SEQ ID NO: 23 - protein, 235 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 24 - protein, 690 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 25 - protein, 214 aa, synthetic construct
DIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 5 aa, synthetic construct
TYWLG

SEQ ID NO: 27 - protein, 17 aa, synthetic construct
IMSPVDSDIRYSPSFQG

SEQ ID NO: 28 - protein, 10 aa, synthetic construct
RRPGQGYFDF

SEQ ID NO: 29 - protein, 11 aa, synthetic construct
RASQGISSWLA

SEQ ID NO: 30 - protein, 7 aa, synthetic construct
AASSLQS

SEQ ID NO: 31 - protein, 9 aa, synthetic construct
QQYNIYPYT

SEQ ID NO: 32 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 33 - protein, 106 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEI

SEQ ID NO: 34 - protein, 445 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 707 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSEPKSSDKTHTEVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSGGGGSGGGGSGGGGSGGGGSDIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEIK

SEQ ID NO: 36 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 104 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 38 - protein, 101 aa, synthetic construct
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 39 - protein, 205 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 40 - protein, 667 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEPKSSDKTHTSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 41 - protein, 5 aa, synthetic construct
DYYMS

SEQ ID NO: 42 - protein, 17 aa, synthetic construct
YISSSGSSIYYADSVKG

SEQ ID NO: 43 - protein, 14 aa, synthetic construct
DRVMTGTTEGDFDY

SEQ ID NO: 44 - protein, 123 aa, synthetic construct
QVQLVESGGGLVKPGGSLRLSCAASGFTFSDYYMSWIRQAPGKGLEWVSYISSSGSSIYYADSVKGRFTISRDNAKNSLYLQMNSLRAEDTAMYYCARDRVMTGTTEGDFDYWGQGTLVTVSS

SEQ ID NO: 45 - protein, 584 aa, synthetic construct
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSEPKSSDKTHTQVQLVESGGGLVKPGGSLRLSCAASGFTFSDYYMSWIRQAPGKGLEWVSYISSSGSSIYYADSVKGRFTISRDNAKNSLYLQMNSLRAEDTAMYYCARDRVMTGTTEGDFDYWGQGTLVTVSS

SEQ ID NO: 46 - protein, 20 aa, synthetic construct
GGGGSGGGGSGGGGSGGGGS

SEQ ID NO: 47 - protein, 5 aa, synthetic construct
DYAMH

SEQ ID NO: 48 - protein, 17 aa, synthetic construct
AITWNSGHIDYADSVEG

SEQ ID NO: 49 - protein, 12 aa, synthetic construct
VSYLSTASSLDY

SEQ ID NO: 50 - protein, 11 aa, synthetic construct
RASQGIRNYLA

SEQ ID NO: 51 - protein, 7 aa, synthetic construct
AASTLQS

SEQ ID NO: 52 - protein, 9 aa, synthetic construct
QRYNRAPYT

SEQ ID NO: 53 - protein, 121 aa, synthetic construct [sequence omitted]

SEQ ID NO: 54 - protein, 107 aa, synthetic construct [sequence omitted]

SEQ ID NO: 55 - protein, 5 aa, synthetic construct
NHWMN

SEQ ID NO: 56 - protein, 19 aa, synthetic construct
EIRSKSINSATHYAESVKG

SEQ ID NO: 57 - protein, 9 aa, synthetic construct
NYYGSTYDY

SEQ ID NO: 58 - protein, 11 aa, synthetic construct
RASQFVGSSIH

SEQ ID NO: 59 - protein, 7 aa, synthetic construct
YASESMS

SEQ ID NO: 60 - protein, 9 aa, synthetic construct
QQSHSWPFT

SEQ ID NO: 61 - protein, 120 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 62 - protein, 107 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 63 - protein, 5 aa, synthetic construct
SYAMH

SEQ ID NO: 64 - protein, 17 aa, synthetic construct
FMSYDGSNKKYADSVKG

SEQ ID NO: 65 - protein, 17 aa, synthetic construct
DRGIAAGGNYYYYGMDV

SEQ ID NO: 66 - protein, 11 aa, synthetic construct
RASQSVYSYLA

SEQ ID NO: 67 - protein, 7 aa, synthetic construct
DASNRAT

SEQ ID NO: 68 - protein, 10 aa, synthetic construct
QQRSNWPPFT

SEQ ID NO: 69 - protein, 126 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 70 - protein, 111 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTV

SEQ ID NO: 71 - protein, 5 aa, synthetic construct
NYWIG

SEQ ID NO: 72 - protein, 17 aa, synthetic construct
IIDPSNSYTRYSPSFQG

SEQ ID NO: 73 - protein, 8 aa, synthetic construct
WYYKPFDV

SEQ ID NO: 74 - protein, 14 aa, synthetic construct
TGSSSNIGSGYDVH

SEQ ID NO: 75 - protein, 7 aa, synthetic construct
GNSKRPS

SEQ ID NO: 76 - protein, 11 aa, synthetic construct
ASWTDGLSLVV

SEQ ID NO: 77 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 78 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 79 - protein, 10 aa, synthetic construct
GYTFTDQTIH

SEQ ID NO: 80 - protein, 17 aa, synthetic construct
YIYPRDDSPKYNENFKG

SEQ ID NO: 81 - protein, 11 aa, synthetic construct
PDRSGYAWFIY

SEQ ID NO: 82 - protein, 11 aa, synthetic construct
KASRDVAIAVA

SEQ ID NO: 83 - protein, 7 aa, synthetic construct
WASTRHT

SEQ ID NO: 84 - protein, 9 aa, synthetic construct
HQYSSYPFT

SEQ ID NO: 85 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 86 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 87 - protein, 10 aa, synthetic construct
GYIFITYWMT

SEQ ID NO: 88 - protein, 17 aa, synthetic construct
QIFPASGSADYNEKFEG

SEQ ID NO: 89 - protein, 7 aa, synthetic construct
GGGGFAY

SEQ ID NO: 90 - protein, 11 aa, synthetic construct
RTSENIYSYLA

SEQ ID NO: 91 - protein, 7 aa, synthetic construct
NAKTLAE

SEQ ID NO: 92 - protein, 9 aa, synthetic construct
QHHYGIPFT

SEQ ID NO: 93 - protein, 116 aa, synthetic construct [sequence omitted]

SEQ ID NO: 94 - protein, 108 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKR

SEQ ID NO: 95 - protein, 5 aa, synthetic construct
GGGGS

SEQ ID NO: 96 - protein, 50 aa, synthetic construct
  REGION 6..50: note = Up to nine copies of the GGGGS repeat sequence could be deleted
GGGGSGGGGSGGGGSGGGGSGGGGSGGGGSGGGGSGGGGSGGGGSGGGGS

SEQ ID NO: 97 - protein, 6 aa, synthetic construct
HHHHHH

(19) United States
(12) Patent Application Publication
(10) Pub. No.: US 2026/0226178 A1
(43) Pub. Date: Aug. 6, 2026

(54) Fusion Proteins Comprising a Tie2 Binding Domain

(71) Applicant: INGENIA Therapeutics, Inc., Watertown, MA (US)

(72) Inventors: Chander S. Peddaboina, Medway, MA (US); Philip Durost, Auburn, MA (US); Jinsam You, Reading, MA (US); Sangyeul Han, Woburn, MA (US)

(73) Assignee: INGENIA Therapeutics, Inc., Watertown, MA (US)

(21) Appl. No.: 19/428,756

(22) Filed: Dec. 22, 2025

Related U.S. Application Data
(60) Provisional application No. 63/738,207, filed on Dec. 23, 2024.

(51) Int. Cl. C07K 16/28; C07K 14/705; C07K 14/71; C07K 16/18; C07K 16/22; C07K 16/24

(52) U.S. Cl. CPC C07K 16/2863; C07K 14/70578; C07K 14/71; C07K 16/18; C07K 16/22; C07K 16/241; C07K 16/244; C07K 2317/31; C07K 2317/52; C07K 2317/565; C07K 2317/569; C07K 2317/622; C07K 2317/76; C07K 2317/77; C07K 2317/92; C07K 2319/00

(57) ABSTRACT

The present disclosure refers to fusion proteins having an antibody against Tie2 or antigen-binding fragment thereof and a soluble target ligand binding domain that binds TNFα, IL-23, VEGF-A, or C5a. The present disclosure also provides methods for making the fusion proteins, and pharmaceutical compositions and methods for preventing or treating angiogenic diseases or regulating angiogenesis, endothelial signaling, inflammation, and/or autoimmune diseases, utilizing the fusion proteins.

### CROSS-REFERENCE TO RELATED APPLICATION

This application claims priority to U.S. Application No. 63/738,207, filed Dec. 23, 2024, the entire contents of which are incorporated by reference herein for all purposes.

### REFERENCE TO ELECTRONIC SEQUENCE LISTING

This application contains a Sequence Listing which has been submitted electronically in XML format and is hereby incorporated by reference in its entirety. Said.XML copy, created on Dec. 18, 2025, is named “01262-0008-00PCT-ST26” and is 95,453 bytes in size.

### FIELD

The present disclosure relates to fusion proteins comprising an anti-Tie2 antibody or an antigen-binding fragment thereof and a second antigen-binding domain that binds tumor necrosis factor alpha (TNFα), interleukin-23 (IL-23), vascular endothelial growth factor-A (VEGF-A), or complement component 5a (C5a). The fusion proteins can be used to facilitate targeted degradation of TNF-α, IL-23, VEGF-A, or C5a.

### INTRODUCTION AND SUMMARY

Angiogenesis occurs dynamically by a variety of regulatory factors during the development, growth, maintenance, and homeostasis of an organism. Blood vessels newly formed in this process act as transport channels for various biomaterials such as nutrients, oxygen, and hormones in the surrounding cells. Functionally and structurally abnormal blood vessels are the direct or indirect cause for the initiation and progression of various diseases. Tumor blood vessels aggravate hypoxia due to their defective function and structure, resulting in tumor progression and metastasis to other tissues, and also in the poor delivery of anticancer drugs into the core of the tumor mass. Defective blood vessels are also found in other various diseases and conditions, in addition to cancer. Examples thereof include various ocular diseases (e.g., diabetic macular edema, wet age-related macular degeneration), viral infections, and acute inflammatory responses such as sepsis. Thus, if a therapeutic agent capable of normalizing pathologic blood vessels is available, it can be applied to the treatment of various patients with vascular abnormalities.

The angiopoietin family plays an important role in the formation and maintenance of blood vessels, and is comprised of four angiopoietins (Ang1, Ang2, Ang3, and Ang4). Angiopoietin-1 (Ang1) binds to the Tie2 receptor present on the surface of vascular endothelial cells to phosphorylate and activate Tie2 receptor, resulting in stabilization of blood vessels. On the other hand, angiopoietin-2 (Ang2) binds to the Tie2 receptor, but acts as an antagonist to induce inactivation of the Tie2 receptor, resulting in destabilization of blood vessels and leakage of blood vessels. It was reported that the expression level of Ang2 is highly increased in the blood of cancer patients, ocular diseases, viral and bacterial infections and inflammatory diseases (Saharinen P et al., 2017, Nature Review Drug Discovery 16:635-661). However, Ang2 is also known to act as an agonist to induce activation of the Tie2 receptor in several processes, including lymphatic tube formation and maintenance, and thus it is believed that Ang2 performs various functions depending on the context.

Up to now, development and clinical testing of various Anti-Ang2 antibodies have been of interest to many biopharmaceutical companies (e.g., U.S. Pat. Nos. 7,658,924, and 8,987,420). These Ang2 antibodies are reported to inhibit the binding of Ang2 to Tie2 and the Ang2 neutralizing effect was reported to hinder the formation of new blood vessels. The anti-angiogenic and anti-cancer activities of these anti Ang2-antibodies have been demonstrated in many preclinical models, and diverse anti-Ang2 antibodies are being clinically tested in various cancer patients. However, their anti-cancer efficacy has been demonstrated to be insufficient. For example, Phase 3 clinical trials conducted by Amgen showed that the anti-cancer efficacy of the Ang2 antibody in ovarian cancer patients was insignificant (Marth C et al., 2017, Eur. J. Cancer, 70:111-121). In addition to cancer models, an Ang2 neutralizing antibody, Nesvacumab, was tested in ocular patients, however it failed to improve upon the efficacy of Eylea® (anti-vascular endothelial growth factor or anti-VEGF) in a clinical phase 2 study. See, e.g., “Regeneron provides update on Eylea® (aflibercept) injection and nesvacumab (ang2 antibody) combination program” press release available at: investor.regeneron.com/news-releases/news-release-details/regeneron-provides-update-eylear-aflibercept-injection- and.

In contrast to the above-mentioned Ang2 neutralization approach, direct Tie2 activation has been also considered as an alternative approach to inhibit angiogenesis and suppress vascular permeability. Recombinant proteins, which bind directly to the Tie2 receptor and induce phosphorylation and activation of Tie2, have also been developed and tested in many preclinical cancer and ocular models. Examples thereof include cartilage oligomeric matrix protein (COMP)-Angl (Cho et al., 2004, PNAS 101 (15): 5547-5552) and Vasculotide (David S et al., 2011, Am J Physiol Lung Cell Mol Physiol 300 (6): L851-L862). Although these agents showed anti-angiogenic and anti-permeability activity, these tend to have very short half-lives and unstable physicochemical properties. In addition, a small molecule compound (AKB-9778) was developed as an inhibitor for a phosphatase, VE-PTP, which inactivates Tie2 by removing a phosphate group from phosphorylated Tie2 (Goel S et al., 2013, J Natl Cancer Inst 105 (16): 1188-1201). This compound indirectly increases Tie2 activity by inhibiting VE-PTP, although it has the disadvantage of activating other receptors as well. See, e.g., Frye M. et al., 2015, J Exp. Med., 212 (13): 2267-2287; Hayashi M, et al., 2013, Nature Communication, 4:1672; and Mellberg S. et al., 2009, FASEB J., 23 (5): 1490-1502). In addition, agonistic Tie2 antibodies have been developed. See, e.g., U.S. Pat. No. 6,365,154B1; and US Patent Publication No. 20170174789A1. These antibodies increased the survival of endothelial cells and inhibited the vascular leakage. Interestingly, herbal extracts were shown to activate Tie2 activity and claimed to be used as skin care cosmetics. See, e.g., Japanese Patent Application Nos. JP2011102273A, JP2018043949A, JP2015168656A.

Tie2 is a receptor protein that promotes the differentiation and stabilization of blood vessels and is highly expressed in blood vessels. If activated, the Tie2 receptor stabilizes blood vessels and it becomes possible to gather surrounding support cells. For example, activated Tie2 in cancer blood vessels normalizes the cancer vessels, eliminating the increased hypoxia within the tumor, supplying the sufficient oxygen by increasing blood flow into the tumor, and increasing the delivery of other anticancer drugs and the penetration of immune cells.

Additionally, like other tyrosine kinase receptors, upon activation by Ang1 or Ang2 ligands, the Tie2 receptor is internalized and degraded in the cell via lysosomal degradation and the bound ligand is released extracellularly (Bogdanovic et al., J Cell Sci, 119 (17): 3551-3560). Thus, direct activation of Tie2 with anti-Tie2 antibodies can promote internalization of the receptor. By increasing the affinity of the antibody for the Tie2 receptor, it may be possible to direct internalization of the anti-Tie2 antibody as well as the Tie2 receptor. The anti-Tie2 antibody, 3H7, was previously disclosed in U.S. Pat. No. 12,024,562, and binds Tie2 with high affinity.

Soluble target ligands include cytokines, growth factors, and complement components. Soluble target ligands have been explored recently as drug targets for the treatment of autoimmune diseases, cancer, and ophthalmological diseases (See Attwood et al., Nat Rev Drug Discov. 19:695-710 (2020)). Common therapeutics targeting soluble ligands include antibodies and decoy receptors.

The present disclosure provides fusion proteins that can bind both Tie2 and a soluble target ligand, forming a tertiary fusion protein-target ligand-Tie2 complex that is internalized into the cell, where the target ligand is degraded. This mechanism of action could provide targeted elimination of soluble ligands from the body.
Embodiment 1 is a fusion protein comprising (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to tumor necrosis factor alpha (TNFα), interleukin 23 (IL-23), or complement component 5a (C5a).
Embodiment 2 is the fusion protein of embodiment 1, wherein the anti-Tie2 antibody or antigen-binding fragment thereof comprises a heavy chain variable region comprising heavy chain CDRs comprising the amino acid sequences of SEQ ID NOs: 7-9 and a light chain variable region comprising light chain CDRs comprising the amino acid sequences of SEQ ID NOs: 10-12.
Embodiment 3 is the fusion protein of embodiment 1 or 2, wherein the anti-Tie2 antibody or antigen-binding fragment thereof comprises a heavy chain variable region comprising a sequence having at least 80%, 85%, 90%, 95%, 96%, 97%, 98%, or 99% identity to the amino acid sequence of SEQ ID NO: 13; and/or a light chain variable region comprising a sequence having at least 80%, 85%, 90%, 95%, 96%, 97%, 98%, or 99% identity to the amino acid sequence of SEQ ID NO: 14.
Embodiment 4 is the fusion protein of any one of embodiments 1-3, wherein the anti-Tie2 antibody or antigen-binding fragment thereof comprises a heavy chain variable region comprising the amino acid sequence of SEQ ID NO: 13; and/or a light chain variable region comprising the amino acid sequence of SEQ ID NO: 14.
Embodiment 5 is the fusion protein of any one of embodiments 1-4, wherein the second antigen-binding domain is a soluble target ligand binding domain.
Embodiment 6 is the fusion protein of the immediately preceding embodiment, wherein the soluble target ligand binding domain comprises an antibody or antigen-binding fragment thereof, or a receptor domain, optionally a receptor extracellular domain.
Embodiment 7 is the fusion protein of any one of embodiments 1-6, wherein the second antigen-binding domain comprises a TNFα binding domain.
Embodiment 8 is the fusion protein of the immediately preceding embodiment, wherein the TNFα binding domain comprises a TNFα receptor domain.
Embodiment 9 is the fusion protein of embodiment 8, wherein the TNFα receptor domain comprises a sequence having at least 80%, 85%, 90%, 95%, 96%, 97%, 98%, or 99% identity to the amino acid sequence of SEQ ID NO: 23.
Embodiment 10 is the fusion protein of embodiment 8 or embodiment 9, wherein the TNFα receptor domain comprises the amino acid sequence of SEQ ID NO: 23.
Embodiment 11 is the fusion protein of embodiment 7, wherein the TNFα binding domain comprises an anti-TNFα antibody or antigen-binding fragment thereof.
Embodiment 12 is the fusion protein of embodiment 11, wherein the anti-TNF antibody or antigen-binding fragment thereof comprises a heavy chain variable region comprising heavy chain CDRs comprising the amino acid sequences of SEQ ID NOs: 47-49 and a light chain variable region comprising light chain CDRs comprising the amino acid sequences of SEQ ID NOs: 50-52.
Embodiment 13 is the fusion protein of any one of embodiments 11-12, wherein the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 53; and/or a light chain variable region comprising an amino acid sequence having at least 80%, 85%, 90%, 95%, 96%, 97%, 98%, or 99% identity to the amino acid sequence of SEQ ID NO: 54.
Embodiment 14 is the fusion protein of any one of embodiments 11-13, wherein the anti-TNFα antibody or antigen-binding fragment thereof comprises a heavy chain variable region comprising the amino acid sequence of SEQ ID NO: 53; and/or a light chain variable region comprising the amino acid sequence of SEQ ID NO: 54.
Embodiment 15 is the fusion protein of embodiment 11, wherein the anti-TNFα antibody or antigen-binding fragment thereof comprises a heavy chain variable region comprising heavy chain CDRs comprising the amino acid sequences of SEQ ID NOs: 55-57 and a light chain variable region comprising light chain CDRs comprising the amino acid sequences of SEQ ID NOs: 58-60.
Embodiment 16 is the fusion protein of embodiment 15, wherein the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 61; and/or a light chain variable region comprising an amino acid sequence having at least 80%, 85%, 90%, 95%, 96%, 97%, 98%, or 99% identity to the amino acid sequence of SEQ ID NO: 62.
Embodiment 17 is the fusion protein of any one of embodiments 15-16, wherein the anti-TNFα antibody or antigen-binding fragment thereof comprises a heavy chain variable region comprising the amino acid sequence of SEQ ID NO: 61; and/or a light chain variable region comprising the amino acid sequence of SEQ ID NO: 62.
Embodiment 18 is the fusion protein of embodiment 11, wherein the anti-TNFα antibody or antigen-binding fragment thereof comprises a heavy chain variable region comprising heavy chain CDRs comprising the amino acid sequences of SEQ ID NOs: 63-65 and a light chain variable region comprising light chain CDRs comprising the amino acid sequences of SEQ ID NOs: 66-68.
Embodiment 19 is the fusion protein of embodiment 18, wherein the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 69; and/or a light chain variable region comprising an amino acid sequence having at least 80%, 85%, 90%, 95%, 96%, 97%, 98%, or 99% identity to the amino acid sequence of SEQ ID NO: 70.
Embodiment 20 is the fusion protein of any one of embodiments 18-19, wherein the anti-TNFα antibody or antigen-binding fragment thereof comprises a heavy chain variable region comprising the amino acid sequence of SEQ ID NO: 69; and/or a light chain variable region comprising the amino acid sequence of SEQ ID NO: 70.
Embodiment 21 is the fusion protein of any one of embodiments 1-6, wherein the second antigen-binding domain comprises an IL-23 binding domain.
Embodiment 22 is the fusion protein of embodiment 21, wherein the IL-23 binding domain is an anti-IL-23 antibody or antigen-binding fragment thereof.
Embodiment 23 is the fusion protein of embodiment 22, wherein the anti-IL-23 antibody or antigen-binding fragment thereof comprises a heavy chain variable region comprising heavy chain CDRs comprising the amino acid sequences of SEQ ID NOs: 26-28 and a light chain variable region comprising light chain CDRs comprising the amino acid sequences of SEQ ID NOs: 29-31.
Embodiment 24 is the fusion protein of any one of embodiments 22-23, wherein the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 32; and/or a light chain variable region comprising an amino acid sequence having at least 80%, 85%, 90%, 95%, 96%, 97%, 98%, or 99% identity to the amino acid sequence of SEQ ID NO: 33.
Embodiment 25 is the fusion protein of any one of embodiments 22-24, wherein the anti-IL-23 antibody or antigen-binding fragment thereof comprises a heavy chain variable region comprising the amino acid sequence of SEQ ID NO: 32; and/or a light chain variable region comprising the amino acid sequence of SEQ ID NO: 33.
Embodiment 26 is the fusion protein of embodiment 22, wherein the anti-IL-23 antibody or antigen-binding fragment thereof comprises a heavy chain variable region comprising heavy chain CDRs comprising the amino acid sequences of SEQ ID NOs: 71-73 and a light chain NOs: 74-76.
Embodiment 27 is the fusion protein of embodiment 26, wherein the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 77; and/or a light chain variable region comprising an amino acid sequence having at least 80%, 85%, 90%, 95%, 96%, 97%, 98%, or 99% identity to the amino acid sequence of SEQ ID NO: 78.
Embodiment 28 is the fusion protein of any one of embodiments 26-27, wherein the anti-IL-23 antibody or antigen-binding fragment thereof comprises a heavy chain variable region comprising the amino acid sequence of SEQ ID NO: 77; and/or a light chain variable region comprising the amino acid sequence of SEQ ID NO: 78.
Embodiment 29 is the fusion protein of embodiment 22, wherein the anti-IL-23 antibody or antigen-binding fragment thereof comprises a heavy chain variable region comprising heavy chain CDRs comprising the amino acid sequences of SEQ ID NOs: 79-81 and a light chain variable region comprising light chain CDRs comprising the amino acid sequences of SEQ ID NOs: 82-84.
Embodiment 30 is the fusion protein of embodiment 29, wherein the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 85; and/or a light chain variable region comprising an amino acid sequence having at least 80%, 85%, 90%, 95%, 96%, 97%, 98%, or 99% identity to the amino acid sequence of SEQ ID NO: 86.
Embodiment 31 is the fusion protein of any one of embodiments 29-30, wherein the anti-IL-23 antibody or antigen-binding fragment thereof comprises a heavy chain variable region comprising the amino acid sequence of SEQ ID NO: 85; and/or a light chain variable region comprising the amino acid sequence of SEQ ID NO: 86.
Embodiment 32 is the fusion protein of embodiment 22, wherein the anti-IL-23 antibody or antigen-binding fragment thereof comprises a heavy chain variable region comprising heavy chain CDRs comprising the amino acid sequences of SEQ ID NOs: 87-89 and a light chain NOs: 90-92.
Embodiment 33 is the fusion protein of embodiment 32, wherein the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 93; and/or a light chain variable region comprising an amino acid sequence having at least 80%, 85%, 90%, 95%, 96%, 97%, 98%, or 99% identity to the amino acid sequence of SEQ ID NO: 94.
Embodiment 34 is the fusion protein of any one of embodiments 32-33, wherein the anti-IL-23 antibody or antigen-binding fragment thereof comprises a heavy chain variable region comprising the amino acid sequence of SEQ ID NO: 93; and/or a light chain variable region comprising the amino acid sequence of SEQ ID NO: 94.
Embodiment 35 is the fusion protein of any one of embodiments 1-6, wherein the second antigen-binding domain comprises a C5a binding domain.
Embodiment 36 is the fusion protein of the immediately preceding embodiment, wherein the C5a binding domain comprises an anti-C5a nanobody.
Embodiment 37 is the fusion protein of embodiment 36, wherein the anti-C5a nanobody comprises a heavy chain variable region comprising heavy chain CDRs comprising the amino acid sequences of SEQ ID NOs: 41-43.
Embodiment 38 is the fusion protein of any one of embodiments 36-37, wherein the anti-C5a nanobody comprises a sequence having at least 80%, 85%, 90%, 95%, 96%, 97%, 98%, or 99% identity to the amino acid sequence of SEQ ID NO: 44.
Embodiment 39 is the fusion protein of any one of embodiments 36-38, wherein the anti-C5a nanobody comprises the amino acid sequence of SEQ ID NO: 44.
Embodiment 40 is a fusion protein comprising (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen binding domain comprising a VEGF-A binding domain, wherein the VEGF-A binding domain comprises a sequence having at least 80%, 85%, 80%, 95%, 96%, 97%, 98%, or 99% identity to the amino acid sequence of SEQ ID NO: 39; further wherein the fusion protein has an IgG4 backbone.
Embodiment 41 is the fusion protein of the immediately preceding embodiment, wherein the VEGF-A binding domain comprises the amino acid sequence of SEQ ID NO: 39.
Embodiment 42 is the fusion protein of any one of embodiments 40-41, wherein the fusion protein comprises one or more mutations in a heavy chain constant region that reduce or eliminate interaction with Fc Receptors.
Embodiment 43 is the fusion protein of embodiment 42, wherein the one or more mutations comprises altering S228, F234, and L235 (EU numbering) to a PAA mutation.
Embodiment 44 is the fusion protein of any one of embodiments 40-43, wherein the fusion protein comprises a CH domain comprising a sequence having at least 80%, 85%, 90%, 95%, 96%, 97%, 98%, or 99% identity to the amino acid sequence of SEQ ID NO: 18.
Embodiment 45 is the fusion protein of any one of embodiments 40-44, wherein the fusion protein comprises a CH domain comprising the amino acid sequence of SEQ ID NO: 18.
Embodiment 46 is the fusion protein of any one of embodiments 40-45, wherein the anti-Tie2 antibody comprises a heavy chain comprising an amino acid sequence having at least 80%, 85%, 90%, 95%, 96%, 97%, 98%, or 99% identity to the amino acid sequence of SEQ ID NO: 19; and/or a light chain comprising an amino acid sequence having at least 80%, 85%, 90%, 95%, 96%, 97%, 98%, or 99% identity to the amino acid sequence of SEQ ID NO: 25.
Embodiment 47 is the fusion protein of any one of embodiments 40-46, wherein the anti-Tie2 antibody comprises a heavy chain comprising the amino acid sequence of SEQ ID NO: 19; and/or a light chain comprising the amino acid sequence of SEQ ID NO: 25.
Embodiment 48 is the fusion protein of any one of the preceding embodiments, wherein the fusion protein comprises a linker between the second antigen-binding domain and the anti-Tie2 antibody or antigen-binding fragment thereof, optionally wherein the linker comprises a sequence having between 5 and 50 amino acid residues, between 10 and 40 residues, between 15 and 30 residues, or 20 residues.
Embodiment 49 is the fusion protein of any one of the preceding embodiments, wherein the fusion protein comprises a linker between the second antigen-binding domain and the anti-Tie2 antibody or antigen-binding fragment thereof, and wherein the linker comprises a sequence having at least 80%, 85%, 90%, 95%, 96%, 97%, 98%, or 99% identity to the amino acid sequence of SEQ ID NO: 21.
Embodiment 50 is the fusion protein of embodiment 49, wherein the fusion protein comprises a linker between the second antigen-binding domain and the anti-Tie2 antibody or antigen-binding fragment thereof, and wherein the linker comprises the amino acid sequence of SEQ ID NO: 21.
Embodiment 51 is the fusion protein of any one of embodiments 1-48, wherein the fusion protein comprises a linker between the second antigen-binding domain and the anti-Tie2 antibody or antigen-binding fragment thereof, and wherein the linker comprises a sequence having at least 80%, 85%, 90%, 95%, 96%, 97%, 98%, or 99% identity to the amino acid sequence of SEQ ID NO: 22.
Embodiment 52 is the fusion protein of the immediately preceding embodiment, wherein the fusion protein comprises a linker between the second antigen-binding domain and the anti-Tie2 antibody or antigen-binding fragment thereof, and wherein the linker comprises the amino acid sequence of SEQ ID NO: 22.
Embodiment 53 is the fusion protein of any one of embodiments 1-39, wherein the anti-Tie2 antigen-binding fragment is an scFv.
Embodiment 54 is the fusion protein of the immediately preceding embodiment, wherein the anti-Tie2 scFv comprises an amino acid sequence having at least 80%, 85%, 90%, 95%, 96%, 97%, 98%, or 99% identity to the amino acid sequence of SEQ ID NO: 15.
Embodiment 55 is the fusion protein of the immediately preceding embodiment, wherein the anti-Tie2 scFv comprises the amino acid sequence of SEQ ID NO: 15.
Embodiment 56 is the fusion protein of any one of embodiments 1-39, wherein the fusion protein has an IgG isotype.
Embodiment 57 is the fusion protein of the immediately preceding embodiment, wherein the IgG isotype is of the IgG1 or IgG4 class.
Embodiment 58 is the fusion protein of the immediately preceding embodiment, wherein the fusion protein comprises one or more mutations in a heavy chain constant region that reduce or eliminate interaction with Fc Receptors.
Embodiment 59 is the fusion protein of the immediately preceding embodiment, wherein the fusion protein has an IgG1 isotype, further wherein the one or more mutations comprises altering L234, L235, G236, and G237 (EU numbering) to a LAGA mutation, a FEGG mutation, an AAGG mutation, an AAGA mutation, a LALA mutation, or a combination thereof.
Embodiment 60 is the fusion protein of the immediately preceding embodiment, wherein the fusion protein comprises a CH domain comprising a sequence having at least 80%, 85%, 90%, 95%, 96%, 97%, 98%, or 99% identity to the amino acid sequence of SEQ ID NO: 16.
Embodiment 61 is the fusion protein of the immediately preceding embodiment, wherein the fusion protein comprises a CH domain comprising the amino acid sequence of SEQ ID NO: 16.
Embodiment 62 is the fusion protein of any one of embodiments 57-61, wherein the anti-Tie2 antibody comprises a heavy chain comprising an amino acid sequence having at least 80%, 85%, 90%, 95%, 96%, 97%, 98%, or 99% identity to the amino acid sequence of SEQ ID NO: 17; and/or a light chain comprising an amino acid sequence having at least 80%, 85%, 90%, 95%, 96%, 97%, 98%, or 99% identity to the amino acid sequence of SEQ ID NO: 25.
Embodiment 63 is the fusion protein of the immediately preceding embodiment, wherein the anti-Tie2 antibody comprises a heavy chain comprising the amino acid sequence of SEQ ID NO: 17; and/or a light chain comprising the amino acid sequence of SEQ ID NO: 25.
Embodiment 64 is the fusion protein of embodiment 58, wherein the fusion protein comprises an IgG4 isotype, and wherein the one or more mutations comprises altering S228, F234, and L235 (EU numbering) to a PAA mutation.
Embodiment 65 is the fusion protein of the immediately preceding embodiment, wherein the fusion protein comprises a CH domain comprising a sequence having at least 80%, 85%, 90%, 95%, 96%, 97%, 98%, or 99% identity to the amino acid sequence of SEQ ID NO: 18.
Embodiment 66 is the fusion protein of the immediately preceding embodiment, wherein the fusion protein comprises a CH domain comprising the amino acid sequence of SEQ ID NO: 18.
Embodiment 67 is the fusion protein of any one of embodiments 64-66, wherein the anti-Tie2 antibody comprises a heavy chain comprising an amino acid sequence having at least 80%, 85%, 90%, 95%, 96%, 97%, 98%, or 99% identity to the amino acid sequence of SEQ ID NO: 19; and/or a light chain comprising an amino acid sequence having at least 80%, 85%, 90%, 95%, 96%, 97%, 98%, or 99% identity to the amino acid sequence of SEQ ID NO: 25.
Embodiment 68 is the fusion protein of the immediately preceding embodiment, wherein the anti-Tie2 antibody comprises a heavy chain comprising the amino acid sequence of SEQ ID NO: 19; and/or a light chain comprising the amino acid sequence of SEQ ID NO: 25.
Embodiment 69 is the fusion protein of any one of the preceding embodiments, wherein the second antigen-binding domain is linked to the C-terminus of the heavy chain (HC) of the anti-Tie2 antibody or antigen-binding fragment thereof.
Embodiment 70 is the fusion protein of any one of the preceding embodiments, wherein the fusion protein comprises, C-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof.
Embodiment 71 is the fusion protein of any one of embodiments 1 to 68, wherein the second antigen-binding domain is linked to the N-terminus of the heavy chain (HC) of the anti-Tie2 antibody or antigen-binding fragment thereof.
Embodiment 72 is the fusion protein of any one of embodiments 1 to 68 or embodiment 71, wherein the fusion protein comprises, N-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof.
Embodiment 73 is the fusion protein of any one of embodiments 1-10, 48-50, 56-63, or 69-70, comprising a heavy chain comprising an amino acid sequence having at least 80%, 85%, 90%, 95%, 96%, 97%, 98%, or 99% identity to the amino acid sequence of SEQ ID NO: 24; and/or a light chain comprising an amino acid sequence having at least 80%, 85%, 90%, 95%, 96%, 97%, 98%, or 99% identity to the amino acid sequence of SEQ ID NO: 25.
Embodiment 74 is the fusion protein of the immediately preceding embodiment, wherein the fusion protein comprises a heavy chain comprising the amino acid sequence of SEQ ID NO: 24; and/or a light chain comprising the amino acid sequence of SEQ ID NO: 25.
Embodiment 75 is a fusion protein comprising a heavy chain comprising an amino acid sequence having at least 80%, 85%, 90%, 95%, 96%, 97%, 98%, or 99% identity to the amino acid sequence of SEQ ID NO: 24; and/or a light chain comprising an amino acid sequence having at least 80%, 85%, 90%, 95%, 96%, 97%, 98%, or 99% identity to the amino acid sequence of SEQ ID NO: 25.
Embodiment 76 is the fusion protein of the immediately preceding embodiment, wherein the fusion protein comprises a heavy chain comprising the amino acid sequence of SEQ ID NO: 24; and/or a light chain comprising the amino acid sequence of SEQ ID NO: 25.
Embodiment 77 is the fusion protein of any one of embodiments 1-6, 21-25, 48, 51-58, 64-66, or 71-72, wherein second antigen-binding domain is an anti-IL-23 antibody, further wherein the anti-IL-23 antibody comprises a heavy chain comprising an amino acid sequence having at least 80%, 85%, 90%, 95%, 96%, 97%, 98%, or 99% identity to the amino acid sequence of SEQ ID NO: 34; and/or a light chain comprising an amino acid sequence having at least 80%, 85%, 90%, 95%, 96%, 97%, 98%, or 99% identity to the amino acid sequence of SEQ ID NO: 36.
Embodiment 78 is the fusion protein the immediately preceding embodiment, wherein the anti-IL-23 antibody comprises a heavy chain comprising the amino acid sequence of SEQ ID NO: 34; and/or a light chain comprising the amino acid sequence of SEQ ID NO: 36.
Embodiment 79 is the fusion protein of any one of embodiments 1-6, 21-25, 48, 51-58, 64-66, 71-72, or 77-78, comprising a heavy chain comprising an amino acid sequence having at least 80%, 85%, 90%, 95%, 96%, 97%, 98%, or 99% identity to the amino acid sequence of SEQ ID NO: 35; and/or a light chain comprising an amino acid sequence having at least 80%, 85%, 90%, 95%, 96%, 97%, 98%, or 99% identity to the amino acid sequence of SEQ ID NO: 36.
Embodiment 80 is the fusion protein of the immediately preceding embodiment, wherein the fusion protein comprises a heavy chain comprising the amino acid sequence of SEQ ID NO: 35; and/or a light chain comprising the amino acid sequence of SEQ ID NO: 36.
Embodiment 81 is a fusion protein comprising a heavy chain comprising an amino acid sequence having at least 80%, 85%, 90%, 95%, 96%, 97%, 98%, or 99% identity to the amino acid sequence of SEQ ID NO: 35 and/or a light chain comprising an amino acid sequence having at least 80%, 85%, 90%, 95%, 96%, 97%, 98%, or 99% identity to the amino acid sequence of SEQ ID NO: 36.
Embodiment 82 is the fusion protein of the immediately preceding embodiment, wherein the fusion protein comprises a heavy chain comprising the amino acid sequence of SEQ ID NO: 35; and/or a light chain comprising the amino acid sequence of SEQ ID NO: 36.
Embodiment 83 is the fusion protein of any one of embodiments 1-6, 40-48, 51-52, 56-58, or 64-70, comprising a heavy chain comprising an amino acid sequence having at least 80%, 85%, 90%, 95%, 96%, 97%, 98%, or 99% identity to the amino acid sequence of SEQ ID NO: 40 and/or a light chain comprising an amino acid sequence having at least 80%, 85%, 90%, 95%, 96%, 97%, 98%, or 99% identity to the amino acid sequence of SEQ ID NO: 25.
Embodiment 84 is the fusion protein of the immediately preceding embodiment, wherein the fusion protein comprises a heavy chain comprising the amino acid sequence of SEQ ID NO: 40; and/or a light chain comprising the amino acid sequence of SEQ ID NO: 25.
Embodiment 85 is a fusion protein comprising a heavy chain comprising an amino acid sequence having at least 80%, 85%, 90%, 95%, 96%, 97%, 98%, or 99% identity to the amino acid sequence of SEQ ID NO: 40 and/or a light chain comprising an amino acid sequence having at least 80%, 85%, 90%, 95%, 96%, 97%, 98%, or 99% identity to the amino acid sequence of SEQ ID NO: 25.
Embodiment 86 is the fusion protein of the immediately preceding embodiment, wherein the fusion protein comprises a heavy chain comprising the amino acid sequence of SEQ ID NO: 40; and/or a light chain comprising the amino acid sequence of SEQ ID NO: 25.
Embodiment 87 is the fusion protein of any one of embodiments 1-6, 35-39, 48, 51-52, 56-58, or 64-70, comprising a heavy chain comprising an amino acid sequence having at least 80%, 85%, 90%, 95%, 96%, 97%, 98%, or 99% identity to the amino acid sequence of SEQ ID NO: 45; and/or a light chain comprising an amino acid sequence having at least 80%, 85%, 90%, 95%, 96%, 97%, 98%, or 99% identity to the amino acid sequence of SEQ ID NO: 25.
Embodiment 88 is the fusion protein of the immediately preceding embodiment, wherein the fusion protein comprises a heavy chain comprising the amino acid sequence of SEQ ID NO: 45; and/or a light chain comprising the amino acid sequence of SEQ ID NO: 25.
Embodiment 89 is a fusion protein comprising a heavy chain comprising an amino acid sequence having at least 80%, 85%, 90%, 95%, 96%, 97%, 98%, or 99% identity to the amino acid sequence of SEQ ID NO: 45; and/or a light chain comprising an amino acid sequence having at least 80%, 85%, 90%, 95%, 96%, 97%, 98%, or 99% identity to the amino acid sequence of SEQ ID NO: 25.
Embodiment 90 is the fusion protein of the immediately preceding embodiment, wherein the fusion protein comprises a heavy chain comprising the amino acid sequence of SEQ ID NO: 45; and/or a light chain comprising the amino acid sequence of SEQ ID NO: 25.
Embodiment 91 is the fusion protein of any one of the preceding embodiments, wherein the fusion protein binds to Tie2 IgG3-FNIII (1-3) domain comprising SEQ ID NOs: 2, 3, or 4 with an affinity KD (M) of about 1E-9 to 1E-12 M.
Embodiment 92 is a pharmaceutical composition comprising the fusion protein of any one of embodiments 1-91 and a pharmaceutically acceptable vehicle, excipient, and/or diluent.
Embodiment 93 is a nucleic acid encoding the fusion protein of any one of embodiments 1-91.
Embodiment 94 is an expression vector comprising the nucleic acid of embodiment 93.
Embodiment 95 is a host cell comprising the nucleic acid of embodiment 93 or the expression vector of embodiment 94.
Embodiment 96 is a fusion protein made by culturing the host cell of embodiment 95 under suitable conditions.
Embodiment 97 is a method of manufacturing the fusion protein of any one of embodiments 1-91 comprising the steps of: culturing the host cell of embodiment 95 under suitable conditions.
Embodiment 98 is the method of the immediately preceding embodiment, further comprising recovering the fusion protein of any one of embodiments 1 to 91 from the cultured cell.
Embodiment 99 is a method of degrading a soluble target ligand comprising administering an effective amount of a fusion protein of any one of embodiments 1-91 or a pharmaceutical composition of embodiment 92.
Embodiment 100 is the method of the immediately preceding embodiment, wherein the fusion protein binds to the soluble target ligand and Tie2 Ig3-FNIII (1-3) to form a fusion protein-target ligand complex, the fusion protein-soluble target ligand complex is internalized by the cell, and subsequently degraded in the lysosome.
Embodiment 101 is the use of the fusion protein of any one of embodiments 1-91 or the pharmaceutical composition of embodiment 92 for the manufacture of a medicament for degrading a soluble target ligand.
Embodiment 102 is the fusion protein of any one of embodiments 1-91 or the pharmaceutical composition of embodiment 92 for use in degrading a soluble target ligand.
Embodiment 103 is a method for reducing the serum concentration of a soluble target ligand in a subject in need thereof, comprising administering an effective amount of the fusion protein of any one of embodiments 1-91 or the pharmaceutical composition of embodiment 92 to a subject in need thereof.
Embodiment 104 is the use of the fusion protein of any one of embodiments 1-91 or the pharmaceutical composition of embodiment 92 for the manufacture of a medicament for reducing the serum concentration of a soluble target ligand in a subject in need thereof.
Embodiment 105 is the fusion protein of any one of embodiments 1-91 or the pharmaceutical composition of embodiment 92 for use in reducing the serum concentration of a soluble target ligand in a subject in need thereof.
Embodiment 106 is a method for preventing or treating an angiogenic disease, comprising administering an effective amount of a fusion protein of any one of embodiments 1-91 or a pharmaceutical composition of embodiment 92 to a subject in need thereof.
Embodiment 107 is the use of the fusion protein of any one of embodiments 1-91 or the pharmaceutical composition of embodiment 92 for the manufacture of a medicament for treating an angiogenic disease in a subject in need thereof.
Embodiment 108 is the fusion protein of any one of embodiments 1-91 or the pharmaceutical composition of embodiment 92 for use in treating an angiogenic disease in a subject in need thereof.
Embodiment 109 is the method, use, fusion protein, or pharmaceutical composition thereof for use of any one of embodiments 106-108, wherein the angiogenic disease is cancer, metastasis, diabetic retinopathy, retinopathy of prematurity, corneal graft rejection, macular degeneration, neovascular glaucoma, systemic erythrosis, proliferative retinopathy, psoriasis, hemophilic arthritis, allied sclerosis, capillary formation of atherosclerotic plaques, keloid, wound granulation, vascular adhesion, rheumatoid arthritis, osteoarthritis, autoimmune diseases, Crohn's disease, restenosis, atherosclerosis, intestinal adhesions, cat scratch disease, ulcer, liver cirrhosis, nephritis, diabetic nephropathy, diabetes mellitus, an inflammatory disease, or a neurodegenerative disease.
Embodiment 110 is the method, use, fusion protein, or pharmaceutical composition thereof for use of embodiment 109, wherein the cancer is esophageal cancer, stomach cancer, large intestine cancer, rectal cancer, oral cancer, pharyngeal cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, or multiple myeloid blood cancer.
Embodiment 111 is the method, use, fusion protein, or pharmaceutical composition thereof for use of embodiment 109, wherein the autoimmune disease is ANCA-associated vasculitis, C3 glomerulopathy, celiac disease, Crohn's disease, diabetes mellitus, focal segmental glomerulosclerosis, IgA nephropathy, inflammatory bowel disease (IBD), lupus nephritis, multiple sclerosis, myasthenia gravis psoriasis, rheumatoid arthritis, systemic lupus erythematosus, or systemic sclerosis.
Embodiment 112 is a method for regulating angiogenesis, endothelial signaling, inflammation, and/or vascular leakage, comprising administering an effective amount of a fusion protein of any one of embodiments 1-91 or a pharmaceutical composition of embodiment 92 to a subject in need thereof.
Embodiment 113 is the method of the immediately preceding embodiment, wherein the inflammation is from sepsis, acute respiratory distress syndromes, and/or virus-infectious diseases.
Embodiment 114 is the method, use, or fusion protein or pharmaceutical composition thereof for use of any one of embodiments 103-113, wherein the subject is a mammal.
Embodiment 115 is the method of embodiment 114, wherein the subject is a cat, a dog, or a human.
Embodiment 116 is the method of embodiment 114, wherein the subject is a human.

### BRIEF DESCRIPTION OF THE DRAWINGS

FIGS. 1A and 1B show fusion proteins composed of a Tie2-binding moiety, a linker, and a second soluble target-binding moiety. Exemplary second targets include TNF-alpha (TNFα), IL-23, VEGF-A, and C5a. FIG. 1A shows the structure of an exemplary fusion protein wherein a decoy receptor, nanobody, or single-chain Fv fragment was added to the C-terminus of the anti-Tie2 antibody. FIG. 1B shows the structure of an exemplary fusion protein wherein a Tie2-binding scFv was added to the C-terminus of the anti-second target antibody.

FIGS. 2A-2D demonstrate flow cytometry measurements of the APC signal generated by binding of the fusion proteins to the biotinylated target and to human Tie2 on CHO-hTie2 cells. FIG. 2A shows ITP-020, the fusion protein for Tie2 and TNF alpha, FIG. 2B shows ITP-218, the fusion protein for Tie2 and IL-23, FIG. 2C shows ITP-221, the fusion protein for Tie2 and VEGF, and FIG. 2D shows ITP-204, the fusion protein for Tie2 and C5a.

FIGS. 3A-3D show the percent internalization of the Tie2 antibodies and various biotinylated targets, as measured in Example 3. FIG. 3A shows TNF alpha-Biotin (ITP-020), FIG. 3B shows IL-23-Biotin (ITP-218), FIG. 3C shows VEGF-A-Biotin (ITP-221), and FIG. 3D shows C5a-Biotin (ITP-204).

### DESCRIPTION OF THE SEQUENCES

TABLE 1
Table 1 provides a listing of certain sequences
referenced herein. Where applicable, CDR sequences are underlined.
Description of the Sequences

| | | | SEQ |
|---|---|---|---|
| Description | Sequence | | ID NO. |
| Human Tie2 | MDSLASLVLCGVSLLLSGTVEGAMDLILINSLPLVSDAETSLTCIASGWR | 50 | 1 |
| Full-Length | PHEPITIGRDFEALMNQHQDPLEVTQDVTREWAKKVVWKREKASKINGAY | 100 | |
| | FCEGRVRGEAIRIRTMKMRQQASFLPATLTMTVDKGDNVNISFKKVLIKE | 150 | |
| | EDAVIYKNGSFIHSVPRHEVPDILEVHLPHAQPQDAGVYSARYIGGNLFT | 200 | |
| | SAFTRLIVRRCEAQKWGPECNHLCTACMNNGVCHEDTGECICPPGFMGRT | 250 | |
| | CEKACELHTFGRTCKERCSGQEGCKSYVFCLPDPYGCSCATGWKGLQCNE | 300 | |
| | ACHPGFYGPDCKLRCSCNNGEMCDRFQGCLCSPGWQGLQCEREGIQRMTP | 350 | |
| | KIVDLPDHIEVNSGKFNPICKASGWPLPTNEEMTLVKPDGTVLHPKDFNH | 400 | |
| | TDHFSVAIFTIHRILPPDSGVWVCSVNTVAGMVEKPFNISVKVLPKPLNA | 450 | |
| | PNVIDTGHNFAVINISSEPYFGDGPIKSKKLLYKPVNHYEAWQHIQVTNE | 500 | |
| | IVTLNYLEPRTEYELCVQLVRRGEGGEGHPGPVRRFTTASIGLPPPRGLN | 550 | |
| | LLPKSQTTLNLTWQPIFPSSEDDFYVEVERRSVQKSDQQNIKVPGNLTSV | 600 | |
| | LLNNLHPREQYVVRARVNTKAQGEWSEDLTAWTLSDILPPQPENIKISNI | 650 | |
| | THSSAVISWTILDGYSISSITIRYKVQGKNEDQHVDVKIKNATITQYQLK | 700 | |
| | GLEPETAYQVDIFAENNIGSSNPAFSHELVTLPESQAPADLGGGKMLLIA | 750 | |
| | ILGSAGMTCLTVLLAFLIILQLKRANVQRRMAQAFQNVREEPAVQFNSGT | 800 | |
| | LALNRKVKNNPDPTIYPVLDWNDIKFQDVIGEGNFGQVLKARIKKDGLRM | 850 | |
| | DAAIKRMKEYASKDDHRDFAGELEVLCKLGHHPNIINLLGACEHRGYLYL | 900 | |
| | AIEYAPHGNLLDFLRKSRVLETDPAFAIANSTASTLSSQQLLHFAADVAR | 950 | |
| | GMDYLSQKQFIHRDLAARNILVGENYVAKIADFGLSRGQEVYVKKTMGRL | 1000 | |
| | PVRWMAIESLNYSVYTTNSDVWSYGVLLWEIVSLGGTPYCGMTCAELYEK | 1050 | |
| | LPQGYRLEKPLNCDDEVYDLMRQCWREKPYERPSFAQILVSLNRMLEERK | 1100 | |
| | TYVNTTLYEKFTYAGIDCSAEEAA | 1124 | |
| Human Tie2 | TPKIVDLPDHIEVNSGKFNPICKASGWPLPTNEEMTLVKPDGTVLHPKDF | 50 | 2 |
| Ig3-FNIII(1-3) | NHTDHFSVAIFTIHRILPPDSGVWVCSVNTVAGMVEKPFNISVKVLPKPL | 100 | |
| | NAPNVIDTGHNFAVINISSEPYFGDGPIKSKKLLYKPVNHYEAWQHIQVT | 150 | |
| | NEIVTLNYLEPRTEYELCVQLVRRGEGGEGHPGPVRRFTTASIGLPPPRG | 200 | |
| | LNLLPKSQTTLNLTWQPIFPSSEDDFYVEVERRSVQKSDQQNIKVPGNLT | 250 | |
| | SVLLNNLHPREQYVVRARVNTKAQGEWSEDLTAWTLSDILPPQPENIKIS | 300 | |
| | NITHSSAVISWTILDGYSISSITIRYKVQGKNEDQHVDVKIKNATITQYQ | 350 | |
| | LKGLEPETAYQVDIFAENNIGSSNPAFSHELVTLPESQAP | 390 | |
| Rabbit Tie2 | TPKIEDLPDHIEVNTGKFNPICKASGWPLPANEEMTLVKPDGTVLHPKDFNHTENFSVAI | 60 | 3 |
| Ig3-FNIII(1-3) | FTIHRILPLDSGVWVCSVNTVAGMVEKPFNISVKVLPKPLNAPNVIDTGHNFAVINISSE | 120 | |
| | PYFGDGPIKSKKLLYKPVNDYEAWRHIQVTNEIVTLNYLEPRTEYELCVQLIRRGEGGEG | 180 | |
| | HPGPVRRFTTASIGLPPPQGLILLPKSQTTLNLTWQPIFPSSEDDFYVEVERRSVQIKSD | 240 | |
| | QQNIKVPGNLTSVLLNNLHPREQYVVRARVNTKAQGEWSEDLTAWTLSDIVPPQPENIKI | 300 | |
| | SNITDSSAVISWTILDGYSISSIIIRYKVQGKNEDQHIDVKIKNATITQYQLKGLEPETA | 360 | |
| | YQVDMFAENNIGSSNPAFSHELMTLPESQAP | 391 | |
| Mouse Tie2 | TPQIEDLPDHIEVNSGKFNPICKASGWPLPTSEEMTLVKPDGTVLQPNDFNYTDRFSVAI | 60 | 4 |
| Ig3-FNIII(1-3) | FTVNRVLPPDSGVWVCSVNTVAGMVEKPFNISVKVLPEPLHAPNVIDTGHNFAIINISSE | 120 | |
| | PYFGDGPIKSKKLFYKPVNQAWKYIEVTNEIFTLNYLEPRTDYELCVQLARPGEGGEGHP | 180 | |
| | GPVRRFTTASIGLPPPRGLSLLPKSQTALNLTWQPIFTNSEDEFYVEVERRSLQTTSDQQ | 240 | |
| | NIKVPGNLTSVLLSNLVPREQYTVRARVNTKAQGEWSEELRAWTLSDILPPQPENIKISN | 300 | |
| | ITDSTAMVSWTIVDGYSISSIIIRYKVQGKNEDQHIDVKIKNATVTQYQLKGLEPETTYH | 360 | |
| | VDIFAENNIGSSNPAFSHELRTLPHSPAS | 389 | |

| | | |
|---|---|---|
| Binding | TLSDILPPQPEN | 5 |
| domain (amino | | |
| acids 633-644 | | |
| of Tie2 of SEQ | | |
| ID NO: 1) | | |
| Binding | FAENNIGSSNPAFS | 6 |
| domain (amino | | |
| acids 713-726 | | |
| of Tie2 of SEQ | | |
| ID NO: 1) | | |
| Antibody 3H7 | SYWMN | 7 |
| CDRH1- | | |
| KABAT | | |
| Antibody 3H7 | MIHPSDSETRLNQKFMD | 8 |
| CDRH2- | | |
| KABAT | | |
| Antibody 3H7 | GLYGNS | 9 |
| CDRH3- | | |
| KABAT | | |
| Antibody 3H7 | RASQDIGISLN | 10 |
| CDRL1- | | |
| KABAT | | |
| Antibody 3H7 | ATSSLDS | 11 |
| CDRL2- | | |
| KABAT | | |
| Antibody 3H7 | LQYASSPYT | 12 |
| CDRL3- | | |
| KABAT | | |
| 3H7 VH (HC | EVQLVQSGAEVKKPGASVKVSCKASGYSFTSYWMNWVRQAPGQGLEWMGMIHPSDSETRLNQKFMDR | 13 |
| Variable | VTMTRDTSTSTVYMELSSLRSEDTAVYYCARGLYGNSWGQGTLVTVSS | |
| Region) | | |
| 3H7 VL (LC | DIQMTQSPSSLSASVGDRVTITCRASQDIGISLNWYQQEPGKAIKRLIYATSSLDSGVPKRFSGSRS | 14 |
| Variable | GTEYTLTISSLESEDFADYYCLQYASSPYTFGGGTKLEI | |
| Region) | | |
| Anti-Tie2 scFv | EVQLVQSGAEVKKPGASVKVSCKASGYSFTSYWMNWVRQAPGQGLEWMGMIHPSDSETRLNQKFMDR | 15 |
| (ITP-218) | VTMTRDTSTSTVYMELSSLRSEDTAVYYCARGLYGNSWGQGTLVTVSSGGGGSGGGGSGGGGSGGGG | |
| | SDIQMTQSPSSLSASVGDRVTITCRASQDIGISLNWYQQEPGKAIKRLIYATSSLDSGVPKRFSGSR | |
| | SGTEYTLTISSLESEDFADYYCLQYASSPYTFGGGTKLEIK | |
| IgG1 LALA | ASTKGPSVFPLAPSSKSTSGGTAALGCLVKDYFPEPVTVSWNSGALTSGVHTFPAVLQSSGLYSLSS | 16 |
| CH domain | VVTVPSSSLGTQTYICNVNHKPSNTKVDKKVEPKSCDKTHTCPPCPAPEAAGGPSVFLFPPKPKDTL | |
| | MISRTPEVTCVVVDVSHEDPEVKFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALHNHYTQKSLSLSPGK | |
| 3H7 VH + | EVQLVQSGAEVKKPGASVKVSCKASGYSFTSYWMNWVRQAPGQGLEWMGMIHPSDSETRLNQKFMDR | 17 |
| IgG1 LALA | VTMTRDTSTSTVYMELSSLRSEDTAVYYCARGLYGNSWGQGTLVTVSSASTKGPSVFPLAPSSKSTS | |
| | GGTAALGCLVKDYFPEPVTVSWNSGALTSGVHTFPAVLQSSGLYSLSSVVTVPSSSLGTQTYICNVN | |
| | HKPSNTKVDKKVEPKSCDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| IgG4 PAA CH | ASTKGPSVFPLAPCSRSTSESTAALGCLVKDYFPEPVTVSWNSGALTSGVHTFPAVLQSSGLYSLSS | 18 |
| domain | VVTVPSSSLGTKTYTCNVDHKPSNTKVDKRVESKYGPPCPPCPAPEAAGGPSVFLFPPKPKDTLMIS | |
| | RTPEVTCVVVDVSQEDPEVQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHNHYTQKSLSLSLG | |
| 3H7 VH + | EVQLVQSGAEVKKPGASVKVSCKASGYSFTSYWMNWVRQAPGQGLEWMGMIHPSDSETRLNQKFMDR | 19 |
| IgG4 PAA | VTMTRDTSTSTVYMELSSLRSEDTAVYYCARGLYGNSWGQGTLVTVSSASTKGPSVFPLAPCSRSTS | |
| | ESTAALGCLVKDYFPEPVTVSWNSGALTSGVHTFPAVLQSSGLYSLSSVVTVPSSSLGTKTYTCNVD | |
| | HKPSNTKVDKRVESKYGPPCPPCPAPEAAGGPSVFLFPPKPKDTLMISRTPEVTCVVVDVSQEDPEV | |
| | QFNWYVDGVEVHNAKTKPREEQFNSTYRVVSVLTVLHQDWLNGKEYKCKVSNKGLPSSIEKTISKAK | |
| | GQPREPQVYTLPPSQEEMTKNQVSLTCLVKGFYPSDIAVEWESNGQPENNYKTTPPVLDSDGSFFLY | |
| | SRLTVDKSRWQEGNVFSCSVMHEALHNHYTQKSLSLSLG | |
| CL1 Domain | KRTVAAPSVFIFPPSDEQLKSGTASVVCLLNNFYPREAKVQWKVDNALQSGNSQESVTEQDSKDSTY | 20 |
| | SLSSTLTLSKADYEKHKVYACEVTHQGLSSPVTKSFNRGEC | |
| (G₄S)₂ Linker | GGGGSGGGGS | 21 |
| IgG₁ Linker | GGGGSGGGGSEPKSSDKTHT | 22 |
| TNFα receptor | LPAQVAFTPYAPEPGSTCRLREYYDQTAQMCCSKCSPGQHAKVFCTKTSDTVCDSCEDSTYTQLWNW | 23 |
| domain | VPECLSCGSRCSSDQVETQACTREQNRICTCRPGWYCALSKQEGCRLCAPLRKCRPGFGVARPGTET | |
| | SDVVCKPCAPGTFSNTTSSTDICRPHQICNVVAIPGNASMDAVCTSTSPTRSMAPGAVHLPQPVSTR | |
| | SQHTQPTPEPSTAPSTSFLLPMGPSPPAEGSTGD | |
| ITP-020 Heavy | EVQLVQSGAEVKKPGASVKVSCKASGYSFTSYWMNWVRQAPGQGLEWMGMIHPSDSETRLNQKFMDR | 24 |
| Chain | VTMTRDTSTSTVYMELSSLRSEDTAVYYCARGLYGNSWGQGTLVTVSSASTKGPSVFPLAPSSKSTS | |
| | GGTAALGCLVKDYFPEPVTVSWNSGALTSGVHTFPAVLQSSGLYSLSSVVTVPSSSLGTQTYICNVN | |
| | HKPSNTKVDKKVEPKSCDKTHTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGKGGGGSGGGGSLPAQVAFTPYAPEP | |
| | GSTCRLREYYDQTAQMCCSKCSPGQHAKVFCTKTSDTVCDSCEDSTYTQLWNWVPECLSCGSRCSSD | |
| | QVETQACTREQNRICTCRPGWYCALSKQEGCRLCAPLRKCRPGFGVARPGTETSDVVCKPCAPGTFS | |
| | NTTSSTDICRPHQICNVVAIPGNASMDAVCTSTSPTRSMAPGAVHLPQPVSTRSQHTQPTPEPSTAP | |
| | STSFLLPMGPSPPAEGSTGD | |
| 3H7 Light | DIQMTQSPSSLSASVGDRVTITCRASQDIGISLNWYQQEPGKAIKRLIYATSSLDSGVPKRFSGSRS | 25 |
| Chain | GTEYTLTISSLESEDFADYYCLQYASSPYTFGGGTKLEIKRTVAAPSVFIFPPSDEQLKSGTASVVC | |
| | LLNNFYPREAKVQWKVDNALQSGNSQESVTEQDSKDSTYSLSSTLTLSKADYEKHKVYACEVTHQGL | |
| | SSPVTKSFNRGEC | |
| Ustekinumab | TYWLG | 26 |
| CDRH1-Kabat | | |
| Ustekinumab | IMSPVDSDIRYSPSFQG | 27 |
| CDRH2-Kabat | | |
| Ustekinumab | RRPGQGYFDF | 28 |
| CDRH3-Kabat | | |
| Ustekinumab | RASQGISSWLA | 29 |
| CDRL1-Kabat | | |
| Ustekinumab | AASSLQS | 30 |
| CDRL2-Kabat | | |
| Ustekinumab | QQYNIYPYT | 31 |
| CDRL3-Kabat | | |
| Ustekinumab | EVQLVQSGAEVKKPGESLKISCKGSGYSFTTYWLGWVRQMPGKGLDWIGIMSPVDSDIRYSPSFQGQ | 32 |
| VH | VTMSVDKSITTAYLQWNSLKASDTAMYYCARRRPGQGYFDFWGQGTLVTVSS | |
| Ustekinumab | DIQMTQSPSSLSASVGDRVTITCRASQGISSWLAWYQQKPEKAPKSLIYAASSLQSGVPSRFSGSGS | 33 |
| VL | GTDFTLTISSLQPEDFATYYCQQYNIYPYTFGQGTKLEI | |
| Ustekinumab | EVQLVQSGAEVKKPGESLKISCKGSGYSFTTYWLGWVRQMPGKGLDWIGIMSPVDSDIRYSPSFQGQ | 34 |
| VH + IgG4 | VTMSVDKSITTAYLQWNSLKASDTAMYYCARRRPGQGYFDFWGQGTLVTVSSASTKGPSVFPLAPCS | |
| PAA | RSTSESTAALGCLVKDYFPEPVTVSWNSGALTSGVHTFPAVLQSSGLYSLSSVVTVPSSSLGTKTYT | |
| | CNVDHKPSNTKVDKRVESKYGPPCPPCPAPEAAGGPSVFLFPPKPKDTLMISRTPEVTCVVVDVSQE | |
| | DPEVQFNWYVDGVEVHNAKTKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLG | |
| ITP-218 heavy | EVQLVQSGAEVKKPGESLKISCKGSGYSFTTYWLGWVRQMPGKGLDWIGIMSPVDSDIRYSPSFQGQ | 35 |
| chain | VTMSVDKSITTAYLQWNSLKASDTAMYYCARRRPGQGYFDFWGQGTLVTVSSASTKGPSVFPLAPCS | |
| | RSTSESTAALGCLVKDYFPEPVTVSWNSGALTSGVHTFPAVLQSSGLYSLSSVVTVPSSSLGTKTYT | |
| | CNVDHKPSNTKVDKRVESKYGPPCPPCPAPEAAGGPSVFLFPPKPKDTLMISRTPEVTCVVVDVSQE | |
| | DPEVQFNWYVDGVEVHNAKTKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGGGGGSGGGGSEPKSSDKTHT | |
| | EVQLVQSGAEVKKPGASVKVSCKASGYSFTSYWMNWVRQAPGQGLEWMGMIHPSDSETRLNQKFMDR | |
| | VTMTRDTSTSTVYMELSSLRSEDTAVYYCARGLYGNSWGQGTLVTVSSGGGGSGGGGSGGGGSGGGG | |
| | SDIQMTQSPSSLSASVGDRVTITCRASQDIGISLNWYQQEPGKAIKRLIYATSSLDSGVPKRFSGSR | |
| | SGTEYTLTISSLESEDFADYYCLQYASSPYTFGGGTKLEIK | |
| ITP-218/ | DIQMTQSPSSLSASVGDRVTITCRASQGISSWLAWYQQKPEKAPKSLIYAASSLQSGVPSRFSGSGS | 36 |
| Ustekinumab | GTDFTLTISSLQPEDFATYYCQQYNIYPYTFGQGTKLEIKRTVAAPSVFIFPPSDEQLKSGTASVVC | |
| Light Chain | LLNNFYPREAKVQWKVDNALQSGNSQESVTEQDSKDSTYSLSSTLTLSKADYEKHKVYACEVTHQGL | |
| | SSPVTKSFNRGEC | |
| VEGFR1 | SDTGRPFVEMYSEIPEIIHMTEGRELVIPCRVTSPNITVTLKKFPLDTLIPDGKRIIWDSRKGFIIS | 37 |
| | NATYKEIGLLTCEATVNGHLYKTNYLTHRQTNTIIDV | |
| VEGFR2 | VLSPSHGIELSVGEKLVLNCTARTELNVGIDFNWEYPSSKHQHKKLVNRDLKTQSGSEMKKFLSTLT | 38 |
| | IDGVTRSDQGLYTCAASSGLMTKKNSTFVRVHEK | |
| VEGF trap | SDTGRPFVEMYSEIPEIIHMTEGRELVIPCRVTSPNITVTLKKFPLDTLIPDGKRIIWDSRKGFIIS | 39 |
| | NATYKEIGLLTCEATVNGHLYKTNYLTHRQTNTIIDVVLSPSHGIELSVGEKLVLNCTARTELNVGI | |
| | DFNWEYPSSKHQHKKLVNRDLKTQSGSEMKKFLSTLTIDGVTRSDQGLYTCAASSGLMTKKNSTFVR | |
| | VHEK | |
| ITP-221 Heavy | EVQLVQSGAEVKKPGASVKVSCKASGYSFTSYWMNWVRQAPGQGLEWMGMIHPSDSETRLNQKFMDR | 40 |
| Chain | VTMTRDTSTSTVYMELSSLRSEDTAVYYCARGLYGNSWGQGTLVTVSSASTKGPSVFPLAPCSRSTS | |
| | ESTAALGCLVKDYFPEPVTVSWNSGALTSGVHTFPAVLQSSGLYSLSSVVTVPSSSLGTKTYTCNVD | |
| | HKPSNTKVDKRVESKYGPPCPPCPAPEAAGGPSVFLFPPKPKDTLMISRTPEVTCVVVDVSQEDPEV | |
| | QFNWYVDGVEVHNAKTKPREEQFNSTYRVVSVLTVLHQDWLNGKEYKCKVSNKGLPSSIEKTISKAK | |
| | GQPREPQVYTLPPSQEEMTKNQVSLTCLVKGFYPSDIAVEWESNGQPENNYKTTPPVLDSDGSFFLY | |
| | SRLTVDKSRWQEGNVFSCSVMHEALHNHYTQKSLSLSLGKGGGGSGGGGSEPKSSDKTHTSDTGRPF | |
| | VEMYSEIPEIIHMTEGRELVIPCRVTSPNITVTLKKFPLDTLIPDGKRIIWDSRKGFIISNATYKEI | |
| | GLLTCEATVNGHLYKTNYLTHRQTNTIIDVVLSPSHGIELSVGEKLVLNCTARTELNVGIDFNWEYP | |
| | SSKHQHKKLVNRDLKTQSGSEMKKFLSTLTIDGVTRSDQGLYTCAASSGLMTKKNSTFVRVHEK | |
| Anti-C5a | DYYMS | 41 |
| CDRH1-Kabat | | |
| Anti-C5a | YISSSGSSIYYADSVKG | 42 |
| CDRH2-Kabat | | |
| Anti-C5a | DRVMTGTTEGDFDY | 43 |
| CDRH3-Kabat | | |
| Anti-C5a | QVQLVESGGGLVKPGGSLRLSCAASGFTFSDYYMSWIRQAPGKGLEWVSYISSSGSSIYYADSVKGR | 44 |
| nanobody | FTISRDNAKNSLYLQMNSLRAEDTAMYYCARDRVMTGTTEGDFDYWGQGTLVTVSS | |
| (clone #85) | | |
| ITP-204 Heavy | EVQLVQSGAEVKKPGASVKVSCKASGYSFTSYWMNWVRQAPGQGLEWMGMIHPSDSETRLNQKFMDR | 45 |
| Chain | VTMTRDTSTSTVYMELSSLRSEDTAVYYCARGLYGNSWGQGTLVTVSSASTKGPSVFPLAPCSRSTS | |
| | ESTAALGCLVKDYFPEPVTVSWNSGALTSGVHTFPAVLQSSGLYSLSSVVTVPSSSLGTKTYTCNVD | |
| | HKPSNTKVDKRVESKYGPPCPPCPAPEAAGGPSVFLFPPKPKDTLMISRTPEVTCVVVDVSQEDPEV | |
| | QFNWYVDGVEVHNAKTKPREEQFNSTYRVVSVLTVLHQDWLNGKEYKCKVSNKGLPSSIEKTISKAK | |
| | GQPREPQVYTLPPSQEEMTKNQVSLTCLVKGFYPSDIAVEWESNGQPENNYKTTPPVLDSDGSFFLY | |
| | SRLTVDKSRWQEGNVFSCSVMHEALHNHYTQKSLSLSLGGGGGSGGGGSEPKSSDKTHTQVQLVESG | |
| | GGLVKPGGSLRLSCAASGFTFSDYYMSWIRQAPGKGLEWVSYISSSGSSIYYADSVKGRFTISRDNA | |
| | KNSLYLQMNSLRAEDTAMYYCARDRVMTGTTEGDFDYWGQGTLVTVSS | |
| (G₄S)₄ Linker | GGGGSGGGGSGGGGSGGGGS | 46 |
| Adalimumab | DYAMH | 47 |
| CDRH1 | | |
| Adalimumab | AITWNSGHIDYADSVEG | 48 |
| CDRH2 | | |
| Adalimumab | VSYLSTASSLDY | 49 |
| CDRH3 | | |
| Adalimumab | RASQGIRNYLA | 50 |
| CDRL1 | | |
| Adalimumab | AASTLQS | 51 |
| CDRL2 | | |
| Adalimumab | QRYNRAPYT | 52 |
| CDRL3 | | |
| Adalimumab | EVQLVESGGGLVQPGRSLRLSCAASGFTFDDYAMHWVRQAPGKGLEWVSAITWNSGHIDYADSVEGR | 53 |
| VH | FTISRDNAKNSLYLQMNSLRAEDTAVYYCAKVSYLSTASSLDYWGQGTLVTVSS | |
| Adalimumab | DIQMTQSPSSLSASVGDRVTITCRASQGIRNYLAWYQQKPGKAPKLLIYAASTLQSGVPSRFSGSGS | 54 |
| VL | GTDFTLTISSLQPEDVATYYCQRYNRAPYTFGQGTKVEIK | |
| Infliximab | NHWMN | 55 |
| CDRH1 | | |
| Infliximab | EIRSKSINSATHYAESVKG | 56 |
| CDRH2 | | |
| Infliximab | NYYGSTYDY | 57 |
| CDRH3 | | |
| Infliximab | RASQFVGSSIH | 58 |
| CDRL1 | | |
| Infliximab | YASESMS | 59 |
| CDRL2 | | |
| Infliximab | QQSHSWPFT | 60 |
| CDRL3 | | |
| Infliximab VH | EVKLEESGGGLVQPGGSMKLSCVASGFIFSNHWMNWVRQSPEKGLEWVAEIRSKSINSATHYAESVK | 61 |
| | GRFTISRDDSKSAVYLQMTDLRTEDTGVYYCSRNYYGSTYDYWGQGTTLTVSS | |
| Infliximab VL | DILLTQSPAILSVSPGERVSFSCRASQFVGSSIHWYQQRTNGSPRLLIKYASESMSGIPSRFSGSGS | 62 |
| | GTDFTLSINTVESEDIADYYCQQSHSWPFTFGSGTNLEVK | |
| Golimumab | SYAMH | 63 |
| CDRH1 | | |
| Golimumab | FMSYDGSNKKYADSVKG | 64 |
| CDRH2 | | |
| Golimumab | DRGIAAGGNYYYYGMDV | 65 |
| CDRH3 | | |
| Golimumab | RASQSVYSYLA | 66 |
| CDRL1 | | |
| Golimumab | DASNRAT | 67 |
| CDRL2 | | |
| Golimumab | QQRSNWPPFT | 68 |
| CDRL3 | | |
| Golimumab | QVQLVESGGGVVQPGRSLRLSCAASGFIFSSYAMHWVRQAPGNGLEWVAFMSYDGSNKKYADSVKGR | 69 |
| VH | FTISRDNSKNTLYLQMNSLRAEDTAVYYCARDRGIAAGGNYYYYGMDVWGQGTTVTVSS | |
| Golimumab | EIVLTQSPATLSLSPGERATLSCRASQSVYSYLAWYQQKPGQAPRLLIYDASNRATGIPARFSGSGS | 70 |
| VL | GTDFTLTISSLEPEDFAVYYCQQRSNWPPFTFGPGTKVDIKRTV | |
| Guselkumab | NYWIG | 71 |
| CDRH1 | | |
| Guselkumab | IIDPSNSYTRYSPSFQG | 72 |
| CDRH2 | | |
| Guselkumab | WYYKPFDV | 73 |
| CDRH3 | | |
| Guselkumab | TGSSSNIGSGYDVH | 74 |
| CDRL1 | | |
| Guselkumab | GNSKRPS | 75 |
| CDRL2 | | |
| Guselkumab | ASWTDGLSLVV | 76 |
| CDRL3 | | |
| Guselkumab | EVQLVQSGAEVKKPGESLKISCKGSGYSFSNYWIGWVRQMPGKGLEWMGIIDPSNSYTRYSPSFQGQ | 77 |
| VH | VTISADKSISTAYLQWSSLKASDTAMYYCARWYYKPFDVWGQGTLVTVSS | |
| Guselkumab | QSVLTQPPSVSGAPGQRVTISCTGSSSNIGSGYDVHWYQQLPGTAPKLLIYGNSKRPSGVPDRFSGS | 78 |
| VL | KSGTSASLAITGLQSEDEADYYCASWTDGLSLVVFGGGTKLTVL | |
| Risankizumab | GYTFTDQTIH | 79 |
| CDRH1 | | |
| Risankizumab | YIYPRDDSPKYNENFKG | 80 |
| CDRH2 | | |
| Risankizumab | PDRSGYAWFIY | 81 |
| CDRH3 | | |
| Risankizumab | KASRDVAIAVA | 82 |
| CDRL1 | | |
| Risankizumab | WASTRHT | 83 |
| CDRL2 | | |
| Risankizumab | HQYSSYPFT | 84 |
| CDRL3 | | |
| Risankizumab | QVQLVQSGAEVKKPGSSVKVSCKASGYTFTDQTIHWMRQAPGQGLEWIGYIYPRDDSPKY | 85 |
| VH | NENFKGKVTITADKSTSTAYMELSSLRSEDTAVYYCAIPDRSGYAWFIYWGQGTLVTVSS | |
| Risankizumab | DIQMTQSPSSLSASVGDRVTITCKASRDVAIAVAWYQQKPGKVPKLLIYWASTRHTGVPS | 86 |
| VL | RFSGSGSRTDFTLTISSLQPEDVADYFCHQYSSYPFTFGSGTKLEIK | |
| Tildrakizumab | GYIFITYWMT | 87 |
| CDRH1 | | |
| Tildrakizumab | QIFPASGSADYNEKFEG | 88 |
| CDRH2 | | |
| Tildrakizumab | GGGGFAY | 89 |
| CDRH3 | | |
| Tildrakizumab | RTSENIYSYLA | 90 |
| CDRL1 | | |
| Tildrakizumab | NAKTLAE | 91 |
| CDRL2 | | |
| Tildrakizumab | QHHYGIPFT | 92 |
| CDRL3 | | |
| Tildrakizumab | QVQLVQSGAEVKKPGASVKVSCKASGYIFITYWMTWVRQAPGQGLEWMGQIFPASGSADYNEKFEGR | 93 |
| VH | VTMTTDTSTSTAYMELRSLRSDDTAVYYCARGGGGFAYWGQGTLVTVSS | |
| Tildrakizumab | DIQMTQSPSSLSASVGDRVTITCRTSENIYSYLAWYQQKPGKAPKLLIYNAKTLAEGVPSRFSGSGS | 94 |
| VL | GTDFTLTISSLQPEDFATYYCQHHYGIPFTFGQGTKVEIKR | |
| G₄S Linker | GGGGS | 95 |
| (G₄S)ₙ Linker | GGGGSGGGGSGGGGSGGGGSGGGGSGGGGSGGGGSGGGGSGGGGSGGGGS | 96 |
| n = 1-10 | (G₄S repeats at amino acid positions 6-50 are optional) | |
| Hexahistidine | HHHHHH | 97 |

### DESCRIPTION OF THE EMBODIMENTS

### I. Definitions

Unless stated otherwise, the following terms and phrases as used herein are intended to have the following meanings:

As used herein, the term “anti-Tie2 antibody” means an antibody specifically binding to Tie2 and includes, in addition to a complete antibody specifically binding to Tie2, antigen-binding fragments of the antibody molecule. In some embodiments, a complete antibody has the structure of two full-length light chains and two full-length heavy chains, and each light chain is connected to a heavy chain by a disulfide bond. The constant region of the heavy chain has gamma (γ), mu (μ), alpha (α), delta (δ) and epsilon (ε) types, with the subclasses of Gamma 1 (γ1), Gamma 2 (γ2), Gamma 3 (γ3), Gamma 4 (γ4), Alpha 1 (α1) and alpha 2 (α2). The constant region of the light chain has kappa (κ) and lambda (2) types.

“Antigen-binding fragments” or “antibody fragments of an antibody” means a fragment which can bind to an antigen, and includes Fab, F(ab′), F(ab′) 2, Fv, and single variable domain on a heavy chain (VHH or nanobody). Among antibody fragments, Fab has one antigen-binding site with a structure of the variable regions of the light and heavy chain, the constant region of the light chain, and the first CHI of the heavy chain.

Fab′ differs from Fab in that it has a hinge region comprising one or more cysteine residues at the C-terminus of the CH1 domain. F(ab′) 2 antibody is produced by disulfide bonds formation between Cysteine residues in the region of the hinge of Fab′. Fv is the smallest antibody fragment having only the variable region of the heavy chain and the variable region of the light chain. Double chain Fv (two-chain Fv) is formed by a non-covalent bond between the heavy chain variable region and the light chain variable region, and single-chain Fv (scFv) is generally formed through a peptide linker covalently between the variable region of the heavy chain and the variable region of the light chain, or is connected directly at the C-terminus by forming a dimer-like structure like a double-chain Fv. This fragment can be obtained by protein hydrolysis enzyme (e.g., one can get Fab by restriction digestion of whole antibody using papain, one can get F(ab′) 2 fragment by cutting with pepsin), also made by genetic manipulation technology.

An antibody may be, for example, in the Fv form (e.g., scFv), or a complete antibody form. In addition, constant region of the heavy chain may be selected from any isotypes of gamma (Y), mu (μ), alpha (α), delta (δ), or epsilon (δ). For example, the constant region is gamma 1 (IgG1), gamma 3 (IgG3), or gamma 4 (IgG4). The light chain constant region can be of kappa or lambda type. An antibody may also be, for example, in the heavy chain only form. The binding fragment of such an antibody is a single variable domain on a heavy chain (VHH).

Antibodies include monoclonal antibodies, multispecific antibodies, human antibodies, humanized antibodies, chimeric antibodies, single chain Fvs (scFv), single chain antibodies, Fab fragments, F(ab′) fragments, Disulfide-binding Fvs (dsFv), heavy chain only antibodies, single variable domain on a heavy chain (VHH) antibodies, nanobodies, and anti-idiotype (anti-Id) antibodies, or of the above antibodies epitope-binding fragments and the like, but are not limited thereto.

The term “nanobody” means a single-domain antibody fragment. In some embodiments, the nanobody is a VHH variable domain.

The term, “heavy chain” or “HC”, means the full-length heavy chain or fragments thereof comprising a variable region domain VH and three constant region domains CH1, CH2, and CH3, having an amino acid sequence with a sufficient variable region in order to provide antigen specificity. In addition, as used herein, “light chain” or “LC”, means the full-length light chain or fragments thereof comprising a variable region domain VL and a constant region domain CL, having an amino acid sequence with a sufficient variable region in order to provide antigen specificity.

A “monoclonal antibody” is an antibody produced by a single clone of cells or cell line and consisting of identical antibody molecules. Monoclonal antibodies can have monovalent affinity, binding only to the same epitope. In contrast, polyclonal antibodies bind to multiple epitopes and are usually made by several different antibody secreting plasma cell lineages. Bispecific monoclonal antibodies can also be engineered, by increasing the targets of one monoclonal antibody to two epitopes.

“Epitope” means a protein determinant to which an antibody can specifically bind to (e.g., the part of an antigen that is recognized by the antibody). An epitope can be a group of chemically active surface molecules, e.g., amino acids or sugar side chains, and generally has a specific charge characteristic as well as a specific three-dimensional structural characteristic.

The “humanized” form of non-human (e.g., murine) antibody is a chimeric antibody comprising one or more amino acid sequence (e.g., one or more CDR sequences, such as 6 CDR sequences) from a non-human antibody (donor or source antibody) and otherwise minimal sequence derived from non-human immunoglobulins. In some embodiments, the humanized antibody is a human immunoglobulin (receptor antibody) whose hypervariable regions are replaced by residues from hypervariable regions of non-human primates, mouse, rat, rabbit or non-human primate (receptor antibody), possessing the desired specificity, affinity and ability of residues from the hypervariable region of the recipient. For humanization one or more residues in the framework domain (FR) can be replaced by the corresponding residue of the non-human donor antibody. This can help to maintain a proper three-dimensional configuration of the grafted CDR(s), thereby improving affinity and antibody stability. Humanized antibodies, e.g., can alternatively or additionally include a new residue that does not appear in the original recipient antibody or donor antibody to further refine additional performance of antibody.

Any “chimeric” antibodies (immunoglobulins) as well as the fragment of the above-mentioned antibody, which exhibit the desired biological activity, are included where part of the heavy and/or light chain derived from a particular species, or identical or homologous to the corresponding sequence in the antibody belonging to the subclass, while the remaining chain(s) are derived from another species, or belonging to other antibody classes or identical to the corresponding sequence in the antibody belonging to the subclass.

“Antibody variable domain” as used herein refers to a domain comprising complementarity determining regions (CDRs; e.g., CDR1, CDR2, and CDR3) and the framework regions (FRs). VH refers to the variable domain of the heavy chain. VL refers to the variable domain of the light chain. “Framework region” (FR) is a variable domain segment outside of the CDRs. Each variable domain typically has 4 FRs identified as FR1, FR2, FR3 and FR4.

“Complementarity determining regions” (CDRs; i.e., CDR1, CDR2, and CDR3) refers to the amino acid residues of the variable domain of the antibody, which are necessary for antigen binding. Each variable domain typically, comprises three CDR regions identified as CDR1, CDR2 and CDR3.

As used herein, “or” has the inclusive sense (i.e., equivalent to and/or) unless the context clearly indicates otherwise.

As used herein, “fusion protein” encompasses any polypeptide comprising sequences from multiple sources. Fusion proteins may be produced, e.g., from a genetic fusion (e.g., a polynucleotide encoding the sequence of the fusion protein) or by chemically joining polypeptides that were produced or synthesized separately. A fusion protein may describe an antibody fused to a second antigen-binding domain, such as an extracellular receptor domain or an antibody or antibody fragment (e.g., an scFv, a VHH variable domain/nanobody, or a full-length antibody).

“Angiogenic disease,” also referred to as “angiogenesis-related disease,” means the occurrence of angiogenesis or a disease associated with progression of angiogenesis.

The term “prevention” as used herein denotes any action to inhibit, delay the onset of, or reduce the likelihood of occurrence of the disease of interest by administering the fusion protein or composition. The terms “treatment” or “therapy” indicate any action that reduces, delays, or mitigates the symptoms of the disease of interest, or cures, reduces the severity of, or delays the progression of the disease of interest.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

As used herein, the term “suitable conditions” for culturing cells indicates conditions which promote cell viability and metabolism, including but not limited to the temperature, percent oxygen saturation, percent CO₂, the and the cell culture medium.

As used herein, the term “about” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### II. Overview

Provided herein are fusion proteins that specifically bind to both Tie2 and a second antigen, wherein the second antigen is TNFα, IL-23, VEGF-A, or C5a. In some embodiments, the fusion proteins can facilitate lysosomal degradation of the second antigen (e.g., lysosomal degradation of TNF-α, IL-23, VEGF-A, or C5a).

### III. Fusion Proteins

### A. Anti-Tie2 Antibodies or Antigen-Binding Fragments

The present disclosure relates to fusion proteins comprising (a) anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to tumor necrosis factor alpha (TNFα), interleukin 23 (IL-23), or complement component 5a (C5a). In some embodiments, the fusion protein comprises (a) anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to VEGF-A. The exemplary second antigen-binding domains of this disclosure are further described in Second Antigen-Binding Domains. In some embodiments, the Tie2 antibody or antigen binding fragment thereof binds to the Tie2 Ig3-FNIII (1-3) domain comprising the sequence of SEQ ID NO: 2. In some embodiments, the Tie2 antibody or antigen-binding fragment thereof includes a heavy chain variable region comprising heavy chain CDR1, CDR2, and CDR3 having the amino acid sequences of SEQ ID NOs: 7-9, respectively, and a light chain variable region comprising light chain CDR1, CDR2, and CDR3 having the amino acid sequences of SEQ ID NOs: 10⁻¹².

In some embodiments, the anti-Tie2 antibody or antigen-binding fragment thereof comprises a heavy chain variable region comprising a sequence having at least 80%, 85%, 90%, 95%, 96%, 97%, 98%, or 99% identity to the amino acid sequence of SEQ ID NO: 13; and/or a light chain variable region comprising a sequence having at least 80%, 85%, 90%, 95%, 96%, 97%, 98%, or 99% identity to the amino acid sequence of SEQ ID NO: 14. In some embodiments, the anti-Tie2 antibody or antigen-binding fragment thereof comprises a heavy chain variable region comprising the amino acid sequence of SEQ ID NO: 13; and/or a light chain variable region comprising the amino acid sequence of SEQ ID NO: 14.

In some embodiments, the anti-Tie2 antibody or antigen-binding fragment thereof is an scFv. In some embodiments, the anti-Tie2 scFv comprises a heavy chain variable region comprising heavy chain CDR1, CDR2, and CDR3 having the amino acid sequences of SEQ ID NOs: 7-9, respectively, and a light chain variable region comprising light chain CDR1, CDR2, and CDR3 having the amino acid sequences of SEQ ID NOs: 10-12. In some embodiments, the anti-Tie2 scFv comprises a heavy chain variable region comprising a sequence having at least 80%, 85%, 90%, 95%, 96%, 97%, 98%, or 99% identity to the amino acid sequence of SEQ ID NO: 13; and/or a light chain variable region comprising a sequence having at least 80%, 85%, 90%, 95%, 96%, 97%, 98%, or 99% identity to the amino acid sequence of SEQ ID NO: 14. In some embodiments, the anti-Tie2 scFv comprises a heavy chain variable region comprising the amino acid sequence of SEQ ID NO: 13; and/or a light chain variable region comprising the amino acid sequence of SEQ ID NO: 14. In some embodiments, the anti-Tie2 scFv comprises a linker between the variable heavy chain and variable light chains (See Linkers). In some embodiments, the anti-Tie2 scFv comprises an amino acid sequence having at least 80%, 85%, 90%, 95%, 96%, 97%, 98%, or 99% identity to the amino acid sequence of SEQ ID NO: 15. In some embodiments, the anti-Tie2 scFv comprises the amino acid sequence of SEQ ID NO: 15.

In some embodiments, the anti-Tie2 antibody has an IgG isotype. In some embodiments, the anti-Tie2 antibody has an IgG1 or IgG4 isotype. In some embodiments, the second antigen-biding domain is an antibody with an IgG isotype. In some embodiments, the second antigen binding domain is an antibody with an IgG1 or IgG4 isotype (See Second Antigen-Binding Domains).

In some embodiments, the fusion protein has an IgG1 isotype, further wherein the one or more mutations comprises altering L234, L235, G236, and G237 (EU numbering) to a LAGA mutation, a FEGG mutation, an AAGG mutation, an AAGA mutation, a LALA mutation, or a combination thereof. In some embodiments, the fusion protein comprises a CH domain comprising a sequence having at least 80%, 85%, 90%, 95%, 96%, 97%, 98%, or 99% identity to the amino acid sequence of SEQ ID NO: 16. In some embodiments, the fusion protein comprises a CH domain comprising the amino acid sequence of SEQ ID NO: 16.

In some embodiments, the fusion protein comprises an IgG4 isotype, and wherein the one or more mutations comprises altering S228, F234, and L235 (EU numbering) to a PAA mutation. In some embodiments, the fusion protein comprises a CH domain comprising a sequence having at least 80%, 85%, 90%, 95%, 96%, 97%, 98%, or 99% identity to the amino acid sequence of SEQ ID NO: 18. In some embodiments, fusion protein comprises a CH domain comprising the amino acid sequence of SEQ ID NO: 18.

Functionally, the binding affinity, or dissociation constant (K_{D}), is the molar (M) concentration of ligand at which half the ligand binding sites on the protein are occupied in the system equilibrium. It is calculated by dividing the dissociation rate (K_{off}) value by the association rate (Kₒₙ) value. In the context of dissociation constant, a lower value is consistent with stronger binding. The K_{D} of the parent 3H7 anti-Tie2 antibody (See U.S. Pat. No. 12,024,562B2, which is herein incorporated by reference in its entirety) with heavy chain CDRs described in SEQ ID NOs: 7-9 and light chain CDRs described in SEQ ID NOs: 10⁻¹², is in the range of 10⁻⁵ M to 10⁻¹² M. In some embodiments, the binding affinity (K_{D}) of the Tie2 antibody or antigen-binding fragment thereof is 10⁻⁶ M to 10⁻¹² M, 10⁻⁷ M to 10⁻¹² M, 10⁸ M to 10⁻¹² M, 10⁻⁹ M to 10⁻¹² M, 10⁻⁵ M to 10⁻¹¹ M, 10⁻⁶ M to 10⁻¹¹ M, 10⁻⁷ M to 10⁻¹¹ M, 10⁻⁸ M to 10⁻¹¹ M, 10⁻⁹ M to 10⁻¹¹ M, 10⁻¹⁰ M to 10⁻¹¹ M, 10⁻⁵ M to 10⁻¹⁰ M, 10⁻⁶ M to 10⁻¹⁰ M, 10⁻⁷ M to 10⁻¹⁰ M, 10⁻⁸ M to 10⁻¹⁰ M, 10⁻⁹ M to 10⁻¹⁰ M, 10⁻⁵ M to 10⁻⁹ M, 10⁻⁶ M to 10⁻⁹ M, 10⁻⁷ M to 10⁻⁹ M, 10⁻⁸ M to 10⁻⁹ M, 10⁻⁵ M to 10⁻⁸ M, 10⁻⁶ M to 10⁻⁸ M, 10⁻⁷ M to 10⁻⁸M, 10⁻⁵ M to 10⁻⁷ M, 10⁻⁶ M to 10⁻⁷ M or 10⁻⁵ M to 10⁻⁶ M.

The exemplary fusion proteins described herein, simultaneously bind Tie2 and a second antigen (e.g., TNF-α, IL-23, VEGF-A, or C5a) to induce internalization of the fusion protein-target-Tie2 complex.

The anti-Tie2 antibody or antibody fragment in the fusion protein can include its biological equivalent within a range that can specifically recognize Tie2. For example, it can include a change to the amino acid sequence to improve further the binding affinity and/or its other biological properties of the antibody. Such modifications can include, for example, deletion, insertions and/or substitutions of amino acid sequence residues of the antibody. These amino acid variations can be made based on the relative similarity of the amino acid substituents, such as hydrophobicity, hydrophilicity, charge, size, etc. of the amino acid side chains, so as to provide conservative substitutions. By the analysis of the size, shape and type of amino acid side chain substituents, it is known that arginine, lysine and histidine are all positively charged residues; alanine, glycine and serine have similar sizes; Phenylalanine, tryptophan and tyrosine have a similar shape. Therefore, based on these considerations, we can say that arginine, lysine and histidine; alanine, glycine and serine; and phenylalanine, tryptophan and tyrosine are conservative substitutions.

Considering the above-described variation having biologically equivalent activity, in some embodiments, the fusion protein comprises an anti-Tie2 antibody, wherein the amino acid sequence of the includes the sequences of the six CDRs in SEQ ID NOs: 7-12, and/or the heavy chains of SEQ ID NO: 17 or 19; and/or the light chain of SEQ ID NO: 25, or any sequence exhibiting substantial identity. Substantial identity means at least 80% identity, such as at least 85% identity, 90% identity, 95% identity, 96% or more, 97% or more, 98% or more, or 99% or more sequence identity.

Based on this, the fusion protein, or components thereof, may have 80%, 85%, 90%, 91%, 92%, 93%, 94%, 95%, 96%, 97%, 98%, 99%, or more identity, when compared to the specified sequence or all sequences described in the specification.

### B. Second Antigen-Binding Domains

Provided herein are fusion proteins that specifically bind to both Tie2 and a second antigen, wherein the second antigen is TNFα, IL-23, VEGF-A, or C5a. In some embodiments, the fusion proteins can facilitate lysosomal degradation of the second antigen (e.g., lysosomal degradation of TNF-α, IL-23, VEGF-A, or C5a).

In some embodiments, the second antigen-binding domain of the fusion protein is a soluble target ligand binding domain. In some embodiments, the soluble target ligand binding domain comprises an antibody or antigen-binding fragment thereof, or a receptor domain, optionally a receptor extracellular domain.

In some embodiments, the fusion protein comprises (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds tumor necrosis factor alpha (TNFα), interleukin 23 (IL-23), or complement component 5a (C5a).

In some embodiments, the fusion protein comprises (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen binding domain comprising a VEGF-A binding domain.

### 1. Tumor Necrosis Factor Alpha (TNFα) Binding Domains

TNFα is an inflammatory cytokine released by immune cells, such as macrophages and T cells (Jang et al., Int J Mol Sci. 22 (5): 2719 (2021)). Excess TNFα is implicated in the pathogenesis of autoimmune diseases, including rheumatoid arthritis (RA), psoriatic arthritis, psoriasis, and inflammatory bowel disease (IBD) such as Crohn's disease (Jang et al. Int J Mol Sci. 22 (5): 2719 (2021); Pagnini C et al., Int J Mol Sci. 22 (19): 10273 (2021)).

Inhibitors of TNFα, such as anti-TNFα antibodies, have been developed for the treatment of autoimmune diseases, in particular, but not limited to rheumatoid arthritis. Such antibodies include Adalimumab (Humira®, Abbvie), Certolizumab (Cimzia®, UCB), Golimumab (Simponi®, Johnson & Johnson), and Infliximab (Remicade®, Johnson & Johnson) (Jang et al. Int J Mol Sci. 22 (5): 2719 (2021); Attwood et al., Nat Rev Drug Discov. 19:695-710 (2020); US Food and Drug Administration, available at fda.gov/drugs/postmarket-drug-safety-information-patients-and-providers/information-tumor-necrosis-factor-tnf-blockers-marketed-remicade-enbrel-humira-cimzia-and-simponi). Another anti-TNFα biologic is etanercept (Enbrel®, Amgen), which is a fusion protein made up of the extracellular domain of the TNFα receptor and an antibody Fc region (See U.S. Pat. No. 8,063,182B).

In some embodiments of the present disclosure, the fusion protein comprises

(a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to tumor necrosis factor alpha (TNFα). In some embodiments, the fusion proteins of the present disclosure comprise a TNFα binding domain. In some embodiments, the TNFα binding domain comprises a TNFα receptor domain. In some embodiments, the TNFα receptor domain is a TNFα receptor extracellular domain. In some embodiments, the TNFα receptor domain is that of etanercept (See U.S. Pat. No. 8,063,182B). In some embodiments, the TNFα receptor domain comprises a sequence having at least 80%, 85%, 90%, 95%, 96%, 97%, 98%, or 99% identity to the amino acid sequence of SEQ ID NO: 23. In some embodiments, the TNFα receptor domain comprises the amino acid sequence of SEQ ID NO: 23.

In some embodiments, the fusion protein comprises a heavy chain comprising an amino acid sequence having at least 80%, 85%, 90%, 95%, 96%, 97%, 98%, or 99% identity to the amino acid sequence of SEQ ID NO: 24; and/or a light chain comprising an amino acid sequence having at least 80%, 85%, 90%, 95%, 96%, 97%, 98%, or 99% identity to the amino acid sequence of SEQ ID NO: 25. In some embodiments, the fusion protein comprises a heavy chain comprising the amino acid sequence of SEQ ID NO: 24; and/or a light chain comprising the amino acid sequence of SEQ ID NO: 25.

The exemplary fusion protein, ITP-020, described herein, comprises an anti-TNFα antibody with a TNFα receptor binding linked to the C-terminus of the anti-Tie2 antibody.

In some embodiments, the TNFα binding domain is an anti-TNFα antibody or antigen-binding fragment thereof. In some embodiments the fusion proteins of the present disclosure comprise an anti-TNFα antibody or antigen-binding fragment thereof.

In some embodiments, the fusion proteins of the present disclosure comprise an anti-TNFα antibody or antigen-binding fragment thereof comprising the variable heavy and light chain regions of Adalimumab (See U.S. Pat. No. 8,663,945). In some embodiments, the anti-TNFα antibody or antigen-binding fragment thereof comprises a heavy chain variable region comprising heavy chain CDRs comprising the amino acid sequences of SEQ ID NOs: 47-49 and a light chain variable region comprising light chain CDRs comprising the amino acid sequences of SEQ ID NOs: 50-52. In some embodiments, the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 53; and/or a light chain variable region comprising an amino acid sequence having at least 80%, 85%, 90%, 95%, 96%, 97%, 98%, or 99% identity to the amino acid sequence of SEQ ID NO: 54. In some embodiments, the anti-TNFα antibody or antigen-binding fragment thereof comprises a heavy chain variable region comprising the amino acid sequence of SEQ ID NO: 53; and/or a light chain variable region comprising the amino acid sequence of SEQ ID NO: 54.

In some embodiments, the fusion proteins of the present disclosure comprise an anti-TNFα antibody or antigen-binding fragment thereof comprising the variable heavy and light chain regions of Infliximab (See U.S. Pat. Nos. 5,656,272 and 6,284,471; see also PDB accession 4g3y). In some embodiments, the anti-TNFα antibody or antigen-binding fragment thereof comprises a heavy chain variable region comprising heavy chain CDRs comprising the amino acid sequences of SEQ ID NOs: 55-57 and a light chain variable region comprising light chain CDRs comprising the amino acid sequences of SEQ ID NOs: 58-60. In some embodiments, the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 61; and/or a light chain variable region comprising an amino acid sequence having at least 80%, 85%, 90%, 95%, 96%, 97%, 98%, or 99% identity to the amino acid sequence of SEQ ID NO: 62. In some embodiments, the anti-TNFα antibody or antigen-binding fragment thereof comprises a heavy chain variable region comprising the amino acid sequence of SEQ ID NO: 61; and/or a light chain variable region comprising the amino acid sequence of SEQ ID NO: 62.

In some embodiments, the fusion proteins of the present disclosure comprise an anti-TNFα antibody or antigen-binding fragment thereof comprising the variable heavy and light chain regions of Golimumab (See US Patent Publication No. 2022/0153827A1). In some embodiments, the anti-TNFα antibody or antigen-binding fragment thereof comprises a heavy chain variable region comprising heavy chain CDRs comprising the amino acid sequences of SEQ ID NOs: 63-65 and a light chain variable region comprising light chain CDRs comprising the amino acid sequences of SEQ ID NOs: 66-68. In some embodiments, the anti-TNFα antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 69; and/or a light chain variable region comprising an amino acid sequence having at least 80%, 85%, 90%, 95%, 96%, 97%, 98%, or 99% identity to the amino acid sequence of SEQ ID NO: 70. In some embodiments, the anti-TNFα antibody or antigen-binding fragment thereof comprises a heavy chain variable region comprising the amino acid sequence of SEQ ID NO: 69; and/or a light chain variable region comprising the amino acid sequence of SEQ ID NO: 70.

In some embodiments, the fusion protein comprises an anti-TNFα antibody, wherein the anti-TNFα antibody has an IgG isotype. In some embodiments, the anti-TNFα antibody has an IgG1 or IgG4 isotype. In some embodiments, the anti-TNFα antibody of the fusion protein has an IgG1 isotype, further wherein the one or more mutations comprises altering L234, L235, G236, and G237 (EU numbering) to a LAGA mutation, a FEGG mutation, an AAGG mutation, an AAGA mutation, a LALA mutation, or a combination thereof. In some embodiments, the fusion protein comprises a CH domain comprising a sequence having at least 80%, 85%, 90%, 95%, 96%, 97%, 98%, or 99% identity to the amino acid sequence of SEQ ID NO: 16. In some embodiments, the fusion protein comprises a CH domain comprising the amino acid sequence of SEQ ID NO: 16.

In some embodiments, the fusion protein comprises an IgG4 isotype, and wherein the one or more mutations comprises altering S228, F234, and L235 (EU numbering) to a PAA mutation. In some embodiments, the fusion protein comprises a CH domain comprising a sequence having at least 80%, 85%, 90%, 95%, 96%, 97%, 98%, or 99% identity to the amino acid sequence of SEQ ID NO: 18. In some embodiments, fusion protein comprises a CH domain comprising the amino acid sequence of SEQ ID NO: 18.

### 2. Interleukin-23 (IL-23) Binding Domains

IL-23 is a member of the IL-2 family of inflammatory cytokines, which also include IL-12, IL-27, and IL-35 (McDonald et al., J Crohns Colitis. 16 (Suppl 2): ii42-ii53 (2022), Krueger et al., Front Immunol. (2024) doi: 10.3389/fimmu.2024.1331217). IL-23 promotes inflammatory diseases, including autoimmune diseases such as inflammatory bowel diseases, including Crohn's disease and ulcerative colitis, psoriasis, and psoriatic arthritis.

Inhibitors of IL-23, such as anti-IL-23 antibodies, have been developed for the treatment of the above-listed autoimmune diseases (Yang et al., Am J Clin Dermatol. 22 (2): 173-192 (2020); McDonald et al., J Crohns Colitis, 16 (Suppl 2): ii42-ii53 (2022)). Examples of such IL-23 antibodies include Guselkumab (Tremfya®, Johnson & Johnson), Risankizumab (Skyrizi®, Abbvie), Tildrakizumab (Ilumya®, Sun Pharma), and Ustekinumab (Stelara®, Johnson & Johnson) (Yang et al., Am J Clin Dermatol. 22 (2): 173-192 (2020); Attwood et al., Nat Rev Drug Discov. 19:695-710 (2020)).

In some embodiments, the fusion protein comprises: (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to interleukin 23 (IL-23).

In some embodiments, the fusion proteins of the present disclosure comprise an IL-23 binding domain. In some embodiments, the fusion proteins of the present disclosure comprise an anti-IL-23 antibody or antigen-binding fragment thereof. In some embodiments, the fusion proteins of the present disclosure comprise an anti-IL-23 antibody or antigen-binding fragment thereof comprising the variable heavy and light chain regions of Ustekinumab (See U.S. Pat. No. 6,902,734B2). In some embodiments, the anti-IL-23 antibody or antigen-binding fragment thereof comprises a heavy chain variable region comprising heavy chain CDRs comprising the amino acid sequences of SEQ ID NOs: 26-28 and a light chain variable region comprising light chain CDRs comprising the amino acid sequences of SEQ ID NOs: 29-31. In some embodiments, the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 32; and/or a light chain variable region comprising an amino acid sequence having at least 80%, 85%, 90%, 95%, 96%, 97%, 98%, or 99% identity to the amino acid sequence of SEQ ID NO: 33. In some embodiments, the anti-IL-23 antibody or antigen-binding fragment thereof comprises a heavy chain variable region comprising the amino acid sequence of SEQ ID NO: 32; and/or a light chain variable region comprising the amino acid sequence of SEQ ID NO: 33.

In some embodiments, the fusion proteins of the present disclosure comprise an anti-IL-23 antibody or antigen-binding fragment thereof comprising the variable heavy and light chain regions of Guselkumab (See U.S. Pat. Nos. 7,935,344, 11,780,911). In some embodiments, the anti-IL-23 antibody or antigen-binding fragment thereof comprises a heavy chain variable region comprising heavy chain CDRs comprising the amino acid sequences of SEQ ID NOs: 71-73 and a light chain variable region comprising light chain CDRs comprising the amino acid sequences of SEQ ID NOs: 74-76. In some embodiments, the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 77; and/or a light chain variable region comprising an amino acid sequence having at least 80%, 85%, 90%, 95%, 96%, 97%, 98%, or 99% identity to the amino acid sequence of SEQ ID NO: 78. In some embodiments, the anti-IL-23 antibody or antigen-binding fragment thereof comprises a heavy chain variable region comprising the amino acid sequence of SEQ ID NO: 77; and/or a light chain variable region comprising the amino acid sequence of SEQ ID NO: 78.

In some embodiments, the fusion proteins of the present disclosure comprise an anti-IL-23 antibody or antigen-binding fragment thereof comprising the variable heavy and light chain regions of Risankizumab (See U.S. Pat. No. 8,778,346). In some embodiments, the anti-IL-23 antibody or antigen-binding fragment thereof comprises a heavy chain variable region comprising heavy chain CDRs comprising the amino acid sequences of SEQ ID NOs: 79-81 and a light chain variable region comprising light chain CDRs comprising the amino acid sequences of SEQ ID NOs: 82-84. In some embodiments, the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 85; and/or a light chain variable region comprising an amino acid sequence having at least 80%, 85%, 90%, 95%, 96%, 97%, 98%, or 99% identity to the amino acid sequence of SEQ ID NO: 86. In some embodiments, the anti-IL-23 antibody or antigen-binding fragment thereof comprises a heavy chain variable region comprising the amino acid sequence of SEQ ID NO: 85; and/or a light chain variable region comprising the amino acid sequence of SEQ ID NO: 86.

In some embodiments, the fusion proteins of the present disclosure comprise an anti-IL-23 antibody or antigen-binding fragment thereof comprising the variable heavy and light chain regions of Tildrakizumab (See U.S. Pat. No. 8,293,883). In some embodiments, the anti-IL-23 antibody or antigen-binding fragment thereof comprises a heavy chain variable region comprising heavy chain CDRs comprising the amino acid sequences of SEQ ID NOs: 87-89 and a light chain variable region comprising light chain CDRs comprising the amino acid sequences of SEQ ID NOs: 90-92. In some embodiments, the anti-IL-23 antibody or antigen-binding fragment thereof comprises a heavy chain variable region comprising an amino acid sequence having at least 80%, 85%, 90%, 95%, 96%, 97%, 98%, or 99% identity to the amino acid sequence of SEQ ID NO: 93; and/or a light chain variable region comprising an amino acid sequence having at least 80%, 85%, 90%, 95%, 96%, 97%, 98%, or 99% identity to the amino acid sequence of SEQ ID NO: 94. In some embodiments, the anti-IL-23 antibody or antigen-binding fragment thereof comprises a heavy chain variable region comprising the amino acid sequence of SEQ ID NO: 93; and/or a light chain variable region comprising the amino acid sequence of SEQ ID NO: 94.

In some embodiments, the fusion protein comprises an anti-IL-23 antibody, wherein the anti-IL-23 antibody has an IgG isotype. In some embodiments, the anti-IL-23 antibody has an IgG1 or IgG4 isotype. In some embodiments, the anti-IL-23 antibody of the fusion protein has an IgG1 isotype, further wherein the one or more mutations comprises altering L234, L235, G236, and G237 (EU numbering) to a LAGA mutation, a FEGG mutation, an AAGG mutation, an AAGA mutation, a LALA mutation, or a combination thereof. In some embodiments, the fusion protein comprises a CH domain comprising a sequence having at least 80%, 85%, 90%, 95%, 96%, 97%, 98%, or 99% identity to the amino acid sequence of SEQ ID NO: 16. In some embodiments, the fusion protein comprises a CH domain comprising the amino acid sequence of SEQ ID NO: 16.

In some embodiments, the fusion protein comprises an IgG4 isotype, and wherein the one or more mutations comprises altering S228, F234, and L235 (EU numbering) to a PAA mutation. In some embodiments, the fusion protein comprises a CH domain comprising a sequence having at least 80%, 85%, 90%, 95%, 96%, 97%, 98%, or 99% identity to the amino acid sequence of SEQ ID NO: 18. In some embodiments, fusion protein comprises a CH domain comprising the amino acid sequence of SEQ ID NO: 18.

In some embodiments, the anti-IL-23 antibody comprises a heavy chain comprising an amino acid sequence having at least 80%, 85%, 90%, 95%, 96%, 97%, 98%, or 99% identity to the amino acid sequence of SEQ ID NO: 34; and/or a light chain comprising an amino acid sequence having at least 80%, 85%, 90%, 95%, 96%, 97%, 98%, or 99% identity to the amino acid sequence of SEQ ID NO: 36. In some embodiments, the anti-IL-23 antibody comprises a heavy chain comprising the amino acid sequence of SEQ ID NO: 34; and/or a light chain comprising the amino acid sequence of SEQ ID NO: 36.

In some embodiments, the fusion protein comprises a heavy chain comprising an amino acid sequence having at least 80%, 85%, 90%, 95%, 96%, 97%, 98%, or 99% identity to the amino acid sequence of SEQ ID NO: 35; and/or a light chain comprising an amino acid sequence having at least 80%, 85%, 90%, 95%, 96%, 97%, 98%, or 99% identity to the amino acid sequence of SEQ ID NO: 36. In some embodiments, the fusion protein comprises a heavy chain comprising the amino acid sequence of SEQ ID NO: 35; and/or a light chain comprising the amino acid sequence of SEQ ID NO: 36.

The exemplary fusion protein, ITP-218, described herein, comprises an anti-IL-23 antibody with an anti-Tie2 scFv linked to the C-terminus of the anti-IL-23 antibody.

### 3. Vascular Endothelial Growth Factor A (VEGF-A) Binding Domains

VEGF-A is a growth factor implicated in vasculogenesis and angiogenesis, as well as the pathophysiology of blood vessel-related diseases such as cancer and ophthalmological diseases Attwood et al., Nat Rev Drug Discov. 19:695-710 (2020); Apte et al., Cell, 176 (6): 1248-1264 (2019)).

Inhibitors of VEGF-A include anti-VEGF-A antibodies, antibody fragments, and decoy receptors, which have been developed for the treatment of cancer and diseases of the eye, including macular oedema, diabetic macular oedema, diabetic retinopathy, and wet age-related macular degeneration (AMD), among others (Attwood et al., Nat Rev Drug Discov. 19:695-710 (2020)). Examples of anti-VEGF-A antibodies and antibody fragments include Bevacizumab (Avastin®, Genentech), Ranibizumab (Lucentis®, Genentech), and Brolucizumab (Beovu®, Novartis). Another VEGF-A-binding biologic is Aflibercept (Eylea®, Regeneron), which is a fusion protein made up of VEGF-receptor binding domains fused to an Fc region of an antibody (See U.S. Pat. No. 7,279,519).

In some embodiments of the present disclosure, the fusion protein comprises (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to vascular endothelial growth factor A (VEGF-A). In some embodiments, the fusion protein comprises a VEGF-A binding domain. In some embodiments, the VEGF-A binding domain comprises an anti-VEGF-A antibody. In some embodiments, the VEGF-A binding domain comprises any one or more VEGF-A receptor extracellular domains. In some embodiments, the VEGF-A binding domain comprises the binding region of VEGF receptor 1 (VEGFR1) of SEQ ID NO: 37 and VEGF receptor 2 (VEGFR2) of SEQ ID NO: 38. In some embodiments, the VEGF-A binding domain comprises a sequence having at least 80%, 85%, 90%, 95%, 96%, 97%, 98%, or 99% identity to the amino acid sequence of SEQ ID NO: 39. In some embodiments, the VEGF-A binding domain comprises the amino acid sequence of SEQ ID NO: 39. In some embodiments, the VEGF-A binding domain comprises the VEGF-A binding domain of Aflibercept (See U.S. Pat. No. 7,279,519).

In some embodiments, the fusion protein comprises (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen binding domain comprising a VEGF-A binding domain, wherein the VEGF-A binding domain comprises a sequence having at least 80%, 85%, 80%, 95%, 96%, 97%, 98%, or 99% identity to the amino acid sequence of SEQ ID NO: 39; further wherein the fusion protein has an IgG4 backbone. In some embodiments, the fusion protein comprises one or more mutations in a heavy chain constant region that reduce or eliminate interaction with Fc Receptors. In some embodiments, the one or more mutations comprises altering S228, F234, and L235 (EU numbering) to a PAA mutation.

In some embodiments, the fusion protein comprises a CH domain comprising a sequence having at least 80%, 85%, 90%, 95%, 96%, 97%, 98%, or 99% identity to the amino acid sequence of SEQ ID NO: 18. In some embodiments, the fusion protein comprises a CH domain comprising the amino acid sequence of SEQ ID NO: 18. In some embodiments, the anti-Tie2 antibody of the fusion protein comprises a heavy chain comprising an amino acid sequence having at least 80%, 85%, 90%, 95%, 96%, 97%, 98%, or 99% identity to the amino acid sequence of SEQ ID NO: 19; and/or a light chain comprising an amino acid sequence having at least 80%, 85%, 90%, 95%, 96%, 97%, 98%, or 99% identity to the amino acid sequence of SEQ ID NO: 25. In some embodiments, the anti-Tie2 antibody of the fusion protein comprises a heavy chain comprising the amino acid sequence of SEQ ID NO: 19; and/or a light chain comprising the amino acid sequence of SEQ ID NO: 25.

In some embodiments, the fusion protein comprises a heavy chain comprising an amino acid sequence having at least 80%, 85%, 90%, 95%, 96%, 97%, 98%, or 99% identity to the amino acid sequence of SEQ ID NO: 40 and/or a light chain comprising an amino acid sequence having at least 80%, 85%, 90%, 95%, 96%, 97%, 98%, or 99% identity to the amino acid sequence of SEQ ID NO: 25. In some embodiments, the fusion protein comprises a heavy chain comprising the amino acid sequence of SEQ ID NO: 40; and/or a light chain comprising the amino acid sequence of SEQ ID NO: 25.

The exemplary fusion protein, ITP-221, described herein, comprises an anti-Tie2 antibody with a VEGF-A receptor domain linked to the C-terminus of the anti-Tie2 antibody.

### 4. Complement Component 5a (C5a) Binding Domains

Complement component 5a (C5a) is a proinflammatory molecule produced by cleavage of complement component 5 (C5) following activation of the complement cascade (Warwick et al., J Biol Chem. 297 (3): 101085 (2021)). Overexpression of C5a leads to inflammation-related disease, including autoimmune diseases such as rheumatoid arthritis, systemic lupus erythematosus, and multiple sclerosis (Schanzenbacher et al., Immunobiology 228 (5): 152413 (2023)).

Inhibitors of C5a include anti-C5 antibodies, which target C5, the precursor to C5a. Examples of such anti-C5 antibodies include Eculizumab (Solaris®, Alexion/AstraZeneca) and Ravulizumab (Ultomiris®, Alexion/AstraZeneca), which have been used for treatment of Paroxysmal nocturnal haemoglobinuria (PNH), atypical haemolytic uraemic syndrome (aHUS), generalized myasthenia gravis, and neuromyelitis optica (Attwood et al., Nat Rev Drug Discov. 19:695-710 (2020)). Vilobelimab (Gohibic™ InflaRx) is an antibody that specifically targets C5a and was recently authorized in 2023 for treatment of COVID-19 in hospitalized adults (US Food and Drug Administration, available at: fda.gov/drugs/drug-safety-and-availability/fda-authorizes-gohibic-vilobelimab-injection-treatment-covid-19).

In some embodiments of the present disclosure, the fusion protein comprises (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to complement component 5a (C5a). In some embodiments, the fusion protein comprises a soluble target ligand binding domain, wherein the soluble target ligand binding domain comprises a complement component 5a (C5a) binding domain.

In some embodiments, the C5a binding domain comprises an anti-C5a antibody or antigen-binding fragment thereof. In some embodiments, the C5a binding domain comprises an anti-C5a nanobody. In some embodiments, the anti-C5a nanobody comprises a heavy chain variable region comprising heavy chain CDRs comprising the amino acid sequences of SEQ ID NOs: 41-43. In some embodiments, the anti-C5a nanobody comprises a sequence having at least 80%, 85%, 90%, 95%, 96%, 97%, 98%, or 99% identity to the amino acid sequence of SEQ ID NO: 44. In some embodiments, the anti-C5a nanobody comprises the amino acid sequence of SEQ ID NO: 44.

In some embodiments, the fusion protein comprises a heavy chain comprising an amino acid sequence having at least 80%, 85%, 90%, 95%, 96%, 97%, 98%, or 99% identity to the amino acid sequence of SEQ ID NO: 45; and/or a light chain comprising an amino acid sequence having at least 80%, 85%, 90%, 95%, 96%, 97%, 98%, or 99% identity to the amino acid sequence of SEQ ID NO: 25. In some embodiments, the fusion protein comprises a heavy chain comprising the amino acid sequence of SEQ ID NO: 45; and/or a light chain comprising the amino acid sequence of SEQ ID NO: 25.

The exemplary fusion protein, ITP-204, described herein, comprises an anti-Tie2 antibody with an anti-C5a nanobody linked to the C-terminus of the anti-Tie2 antibody.

### C. Linkers

In some embodiments, the fusion protein comprises a linker between the second antigen-binding domain and the anti-Tie2 antibody or antigen-binding fragment thereof. In some embodiments, the fusion protein comprises an anti-Tie2 scFv, comprising a linker between the variable heavy and variable light chains.

In some embodiments, the second antigen-binding domain of the fusion protein is linked to the C-terminus of the heavy chain (HC) of the anti-Tie2 antibody or antigen-binding fragment thereof. In some embodiments, the fusion protein comprises, C-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof.

In some embodiments, the second antigen-binding domain of the fusion protein is linked to the N-terminus of the heavy chain (HC) of the anti-Tie2 antibody or antigen-binding fragment thereof. In some embodiments, the fusion protein comprises, N-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof.

In some embodiments, the linker comprises a sequence having between 5 and 50 amino acid residues, between 10 and 40 residues, between 15 and 30 residues, or 20 residues.

In some embodiments, the linker comprises any one or more repeats of the amino acid sequence GGGGS (SEQ ID NO: 95), otherwise referred to as (G4S) n, where n is an integer from 1 to 10 (SEQ ID NO: 96). In some embodiments, the linker comprises the sequence (G4S) 2 (SEQ ID NO: 21). In some embodiments, the linker comprises an amino acid sequence having at least 80%, 80%, 85%, 90%, 95%, 96%, 97%, 98%, or 99% identity to the amino acid sequence of SEQ ID NO: 21. In some embodiments, the linker comprises the amino acid sequence of SEQ ID NO: 21.

In some embodiments, the linker additionally comprises a human IgG1 hinge region (Positions 99-110 of Accession No. P01857). In some embodiments, the linker comprises the sequence (G4S) 2 (SEQ ID NO: 21) and an IgG1 hinge sequence. In some embodiments, the linker comprises an amino acid sequence having at least 80%, 85%, 90%, 95%, 96%, 97%, 98%, or 99% identity to the amino acid sequence of SEQ ID NO: 22. In some embodiments, the linker comprises the amino acid sequence of SEQ ID NO: 22.

In some embodiments, the linker comprises the amino acid sequence (G4S) 4 (SEQ ID NO: 46). In some embodiments, the linker comprises the amino acid sequence of SEQ ID NO: 46.

### IV. Polynucleotides; Methods of Production; Vectors and Host Cells

In another aspect, the present disclosure relates to nucleic acids encoding the fusion proteins described herein.

The fusion protein can be produced recombinantly by constructing or isolating the nucleic acid encoding the fusion protein. Further cloning (DNA amplification) by isolating nucleic acids, and inserting it into a replicable vector may be done or further expression may be made. Based on this, the present disclosure relates to the vector containing the nucleic acid in another aspect.

In some embodiments, the disclosure relates to a nucleic acid comprising the fusion protein. “Nucleic acid” is meant to encompass DNA (gDNA and cDNA) and RNA molecules inclusively, and nucleotide, the basic structural unit of nucleic acid, includes the nucleotide in nature, as well as the analogue with modified sugar or base moieties. The sequence of the nucleic acid encoding the heavy and light chain variable regions can be modified. The modifications include addition, deletion, or non-conservative or conservative substitution of nucleotides.

The DNA encoding the fusion protein is easily separated or synthesized using a conventional process (for example, by using oligonucleotide probes capable of specifically binding to the DNA encoding the heavy and light chains). Many vectors are available. Vector components generally include one or more of the following, but is not limited to: signal sequence, origin of replication, one or more marker genes, enhancer element, promoter, and transcription termination sequence.

In some embodiments, the disclosure provides an expression vector comprising the nucleic acid encoding the fusion protein. In some embodiments, the expression vector further comprises a promoter/enhancer, such as a human cytomegalovirus IE1 (CMV-IE1) promoter/enhancer.

As used herein, the term “vector”, as means for expression a gene of interest in a host cell, includes plasmid vectors, cosmid vector, bacteriophage vector, adenovirus vectors, retroviral vectors and viral vectors such as adeno-associated virus vectors. The nucleic acid encoding the antibody in the vector is operatively linked to a promoter.

As used herein, the terms “operatively linked” or “linked” mean the functional linkage between a nucleic acid expression control sequence (for example, promoter, signal sequence, or array of transcriptional regulator binding sites) and different nucleic acid sequences, Thereby, the control sequence controls transcription and/or translation of the other nucleic acid sequence.

In some embodiments, provided herein is a host cell comprising a polynucleotide encoding a fusion protein described herein. In some embodiments, provided herein is a host cell comprising a vector comprising a polynucleotide encoding a fusion protein described herein. The host cell may be prokaryotic or eukaryotic. The host cell may be isolated, e.g., cultured or not part of a multicellular organism. In some embodiments, the host cell is a member of a cell line. In some embodiments, the host cell is a mammalian cell. In some embodiments, the host cell is an immortalized mammalian cell.

In the case of prokaryotic cell as a host, a strong promotor which can process the transcription (for example, tac promoter, lac promoter, lacUV5 promoter, lpp promoter, pLA promoter, pRA promoter, rac5 promoter, amp promoter, recA promoter, SP6 Promoter, trp promoter and T7 promoter, etc.), ribosome binding site for initiation of translation and transcription/translation termination sequences are generally included. In addition, for example, in the case of eukaryotic cell as a host, a promoter derived from the genome of mammalian cells (example: metallothionine promoter, β-actin promoter, human hemoglobin promoter and human muscle creatine promoter) or a promotor derived from mammalian viruses (example: adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, Moloney Virus promotor, Epstein Barr Virus (EBV) promoter, and Rous sarcoma virus (RSV) promoter) can be used, and a polyadenylation sequence can be included generally as a transcription termination sequence.

In some cases, the vector may be fused with other sequences to facilitate the purification of the antibody expressed. The sequence to be fused is, for example, glutathione S-transferase (Pharmacia, USA), maltose binding protein (NEB, USA), FLAG (IBI, USA) and 6× His (SEQ ID NO: 97) (hexahistidine; Qiagen, USA).

The vector contains an antibiotic resistance gene commonly used in the art as a selection marker, for example a resistant gene to ampicillin, gentamicin, carbenicillin, chloramphenicol, streptomycin, kanamycin, geneticin, neomycin and tetracycline.

In another aspect, the present disclosure relates to a cell transformed with the above-mentioned expression vector. Cells used to produce the antibodies may be prokaryote, yeast and a higher eukaryotic cell, but not limited thereto.

Prokaryotic host cells such as Escherichia coli, Bacillus strains such as Bacillus subtilis and Bacillus thuringiensis, Streptomyces, Pseudomonas (e.g., Pseudomonas putida), Proteus mirabilis and Staphylococcus (for example, Staphylococcus carnosus), can be used.

Exemplary useful animal host cell lines are COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but not limited thereto.

In one embodiment, the disclosure provides a method of manufacturing a fusion protein which binds Tie2 and a second antigen, wherein the second antigen is TNFα, IL-23, VEGF-A, or C5a, comprising the steps of: culturing a cell comprising a nucleic acid, such as an expression vector, encoding the fusion protein (e.g., transformed with an expression vector comprising a nucleic acid encoding the fusion protein); and recovering the fusion protein from the cultured cell.

The cells can be cultured in various media. Any commercial culture media can be used without limitation. All other essential supplements known to those skilled in the art may be included in an appropriate concentration. Culture conditions, such as temperature, pH, etc., and selected host cells are known to those skilled in the art.

Recovery of the fusion protein, antibody, or antigen-binding fragment thereof can be made through removing impurities for example using centrifugation or ultrafiltration, and for example, using affinity chromatography, etc. Additional extra purification technology such as anion or cation exchange chromatography, hydrophobic interaction chromatography, hydroxyapatite chromatography, and the like can be used.

### V. Use of Fusion Proteins

### A. Targeted Degradation

In another aspect, the present disclosure relates to methods and compositions for degrading a soluble target ligand, wherein the soluble target ligand is TNFα, IL-23, VEGF-A or C5a. In some aspects, the method of degrading a soluble target ligand comprises administering an effective amount of any one of the fusion proteins of the present disclosure or pharmaceutical compositions thereof. In some aspects, the fusion protein binds to the soluble target ligand and Tie2 Ig3-FNIII (1-3) to form a fusion protein-soluble target ligand complex, the fusion protein-soluble target ligand complex is internalized by the cell, and subsequently degraded in the lysosome of the cell.

In some aspects, the method of targeted degradation is used to reduce the serum concentration of the soluble target ligand in a subject in need thereof by administering an effective amount of any one of the fusion proteins of the present disclosure or pharmaceutical compositions thereof. In some aspects, the subject is human.

### B. Therapy

In another aspect, the present disclosure relates to methods and compositions for preventing or treating angiogenic diseases by administering an effective amount of the fusion protein. In other aspects, the present embodiments relate to methods and compositions for regulating angiogenesis, endothelial signaling, inflammation, and/or vascular leakage by administering the fusion protein to a subject in need thereof. In some embodiments, a fusion protein described herein is for use in therapy.

Angiogenesis means the formation or growth of new blood vessels from previously existing blood vessels. Examples of angiogenic or angiogenesis-related diseases include, but are not limited to, cancer, metastasis, diabetic retinopathy, retinopathy of prematurity, corneal graft rejection, macular degeneration, neovascular glaucoma, systemic erythrosis, proliferative retinopathy, psoriasis, hemophilic arthritis, capillary formation in atherosclerotic plaques, keloid, wound granulation, vascular adhesion, rheumatoid arthritis, degenerative osteoarthritis, autoimmune diseases, Crohn's disease, restenosis, atherosclerosis, intestinal adhesions, cat scratch disease, ulcers, liver cirrhosis, nephritis, diabetic nephropathy, diabetes mellitus, inflammatory diseases, and neurodegenerative disease. In addition, exemplary cancers include, but are not limited to esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid blood cancer.

In other aspects, the fusion protein may be used for cancer prevention or treatment in an individual in need thereof.

In some embodiments, the fusion protein can prevent the occurrence or progression of cancer by inhibiting angiogenesis and/or removing soluble target ligands from the bloodstream. Examples of the cancer include, but are not limited to, esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid cancer blood cancer.

In some embodiments, the fusion protein may be used for autoimmune disease prevention or treatment in an individual in need thereof. In some embodiments, the fusion protein can prevent the occurrence or progression of autoimmune disease by inhibiting angiogenesis and/or removing soluble target ligands from the bloodstream. Examples of autoimmune disease include ANCA-associated vasculitis, C3 glomerulopathy, celiac disease, Crohn's disease, diabetes mellitus, focal segmental glomerulosclerosis, IgA nephropathy, inflammatory bowel disease (IBD), lupus nephritis, multiple sclerosis, myasthenia gravis psoriasis, rheumatoid arthritis, systemic lupus erythematosus, or systemic sclerosis, but is not limited thereto.

The fusion protein may be administered to regulate angiogenesis, endothelial signaling, inflammation, and/or vascular leakage. For example, the fusion protein may be administered to increase vascular endothelial cellular membrane integrity and/or reduce vascular leakage. Vascular leakage is known to contribute to visual impairment in several common ocular disorders including, but not limited to, diabetic macular edema (DME), diabetic retinopathy (DR) and age-related macular degeneration (AMD). In some embodiments, the inflammation is from sepsis, respiratory distress syndromes, and/or virus-infectious diseases.

The fusion protein, and compositions thereof, may be administered into mammals, including rats, mice, livestock, humans, etc. In some embodiments, the subject is human. Administration is via the typically accepted routes, for example, oral, rectal, intravitreal, intravenous, subcutaneous, intrauterine, or intracerebrovascular administration. In some embodiments, administration is by intravenous injection.

### VI. Pharmaceutical Compositions; Administration

In some embodiments, the composition comprising the fusion protein is a pharmaceutical composition. In some embodiments, the composition includes a suitable vehicle, excipient or diluent typically used in the field.

Pharmaceutical compositions having a pharmaceutically acceptable vehicle can be various oral or parenteral dosage form such as tablets, pills, powders, granules, capsules, suspensions, oral solutions, emulsions, syrups, sterile aqueous solutions, non-aqueous solutions, suspensions, lyophilizates, and suppository. The pharmaceutical composition can include a diluent or excipient, which can be formulated in combination, such as fillers, thickeners, binders, wetting agents, disintegrants, surfactants, etc. Solid preparations for oral administration may be in the form of tablets, pills, powders, granules, capsules, and the like. In connection with the solidarity the compound can be formulated by combining one or more excipients such as starch, calcium carbonate, sucrose, lactose, or gelatin. Simple excipient and lubricating agents such as magnesium stearate, talc, and the like may additionally be used.

The liquid preparation for oral administration may be a suspension, an oral solution, an emulsion, a syrup, or the like. Excipients such as water or simple diluents like wet paraffin, a variety of wetting agents, sweeteners, aromatics, preservatives, and etc. can be included in a liquid formulation. In addition, the pharmaceutical compositions may be in parenteral dosage form such as sterile aqueous solution, non-aqueous solvent, suspension, emulsion, lyophilisate, suppository, etc. Injectable propylene glycol, polyethylene glycol, vegetable oils such as olive oil and esters such as ethyl oleate may be suitable for insoluble solvent and suspension. The basic substance of the suppository includes Witepsol, macrogol, Tween 61, cacao butter, laurin butter and glycerogelatin.

The composition is administered in a pharmaceutically effective amount. Terms used herein, such as a “pharmaceutically effective amount” refers to the enough amount of the pharmaceutical composition for disease treatment, with an appropriate benefit/risk ratio which can be applicable for all medical treatments. The effective amount can be different depending on various factors including parameters like the severity of the disease, the patient's age and sex, type of disease, drug activity, drug sensitivity, administration time, route of administration, secretion rate, duration of treatment, and other factors. Also, the above composition can be administered in single doses or divided into multiple doses. When fully considering these factors, it is important to administer the minimum amount sufficient to obtain maximum effect without side effects. The dosage of the pharmaceutical composition is not particularly limited, but it varies depending on various factors, including patient's health status and weight, disease severity, drug type, administration route and administration time.

The composition may be administered into mammals, including rats, mice, livestock, humans, etc., by one time or multiple times a day, via typically accepted routes. In some embodiments, the composition is administered parenterally. In some embodiments, the composition is administered orally, rectally, intravenously, subcutaneously, intrauterinely, intravitreally, or intracerebrovascularly. In some embodiments, the composition is administered by intravenous injection. In some embodiments, the subject is human.

The present disclosure in other perspective refers to the prevention or treatment methods for angiogenic diseases, and anti-angiogenic methods, including the steps for administering into an individual in need of the fusion protein or the above composition. In some embodiments, the angiogenic disease is an autoimmune disease or cancer (See Therapy).

The fusion proteins can be provided in a pharmaceutical composition.

The methods of the present disclosure include procedures for administering a pharmaceutical composition of pharmaceutically effective dose for individuals in need of inhibition of angiogenesis and/or removal of soluble target ligands from the bloodstream. The individual can be a mammal such as dog, cat, cow, horse, rabbit, mouse, rat, chicken, and human, but it is not limited thereto. The pharmaceutical composition can be administered via a suitable way including parenterally, subcutaneously, intraperitoneally, intrapulmonarily or intranasally, and if necessary, intralesionally for local treatment. In some embodiments, the dosage of the pharmaceutical composition changes depending on various factors including, but not limited to, the health status and weight of the individual, the severity of the disease, the type of drug, the route and time of administration, and it can be easily determined by those skilled in the art.

In other aspects, the present disclosure refers to the methods of cancer prevention or treatment, including administering procedures of the composition (e.g., the fusion protein) to an individual in need thereof for cancer prevention or treatment.

Cancer is not limited as long as it is treatable with the fusion proteins of the present disclosure. Specifically, the fusion protein can prevent the occurrence or progression of cancer by inhibiting angiogenesis and/or removing soluble target ligands from the bloodstream. Examples of the cancer include esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid cancer blood cancer, but is not limited thereto.

In other aspects, the present disclosure refers to the methods of autoimmune disease prevention or treatment, including administering procedures of the composition (e.g., the fusion protein) to an individual in need thereof for autoimmune disease prevention or treatment.

In some embodiments, the fusion protein can prevent the occurrence or progression of autoimmune disease by inhibiting angiogenesis and/or removing soluble target ligands from the bloodstream. Examples of autoimmune disease include ANCA-associated vasculitis, C3 glomerulopathy, celiac disease, Crohn's disease, diabetes mellitus, focal segmental glomerulosclerosis, IgA nephropathy, inflammatory bowel disease (IBD), lupus nephritis, multiple sclerosis, myasthenia gravis psoriasis, rheumatoid arthritis, systemic lupus erythematosus, or systemic sclerosis, but is not limited thereto.

Unless defined otherwise, all technical and scientific terms used herein have the same meaning as those appreciated by those skilled in the field to which the present disclosure pertains.

### Examples

The following examples are intended merely to illustrate the invention and are not constructed to restrict the invention.

### Example 1. Preparation of Fusion Proteins

### A. Construct Design

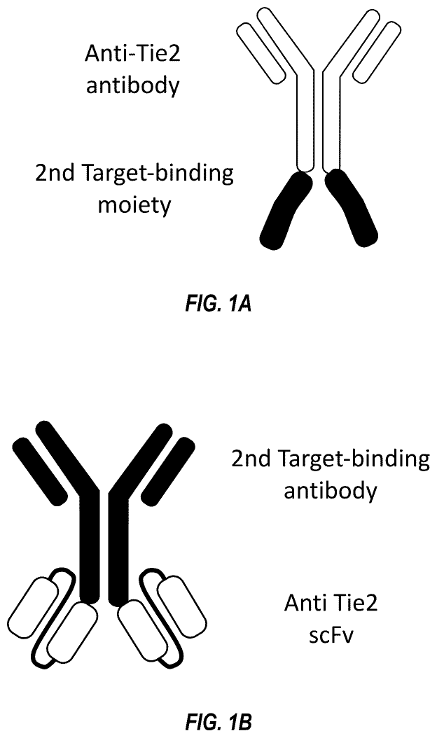

Exemplary fusion proteins were generated by adding a second soluble target-neutralizing component to the C-terminus or N-terminus of a human Tie2-specific antibody or scFv (containing 6 CDRs of antibody 3H7, See U.S. Pat. No. 12,024,562B2) or adding a Tie2-binding scFv to a second target-binding antibody (FIG. 1). The second targets included tumor necrosis factor-alpha (TNF-alpha, TNFα), IL-23, vascular endothelial growth factor-A (VEGF-A), and C5a. The sequences of the heavy and light chains for each fusion protein are shown in Table 1.

To create the anti-TIE2/anti-TNF-alpha bispecific molecule (ITP-020), the TNF-alpha receptor sequence from Etanercept (Enbrel®, See U.S. Pat. No. 8,063,182B) (SEQ ID NO: 23) was incorporated and attached to the C-terminus of the heavy chain of the anti-TIE2 antibody to produce the full heavy chain of SEQ ID NO: 24. This was achieved using a (G4S) 2 linker (SEQ ID NO: 21) with a human IgG1-LALA Fc region (SEQ ID NO: 16). The light chain of ITP-020 sequence is based on that of the 3H7 anti-TIE2 antibody (SEQ ID NO: 25).

To generate the anti-IL23/anti-TIE2 bispecific molecule (ITP-218), the VH and VL sequences of the anti-IL23 antibody, Ustekinumab Stelara® was utilized (See U.S. Pat. No. 6,902,734B2). The scFv of the anti-TIE2 antibody (SEQ ID NO: 15) was attached to the C-terminus of the heavy chain of Ustekinumab (VH is SEQ ID NO: 32, Heavy Chain is SEQ ID NO: 34), utilizing a human IgG4-PAA Fc region (SEQ ID NO: 18) with a 20-mer linker ((G4S) 2 (SEQ ID NO: 21) and a human IgG1 hinge, SEQ ID NO: 22) to produce the full heavy chain of SEQ ID NO: 35. The light chain sequence remains the same as in Ustekinumab (SEQ ID NO: 36). The Ustekinumab CDR sequences (based on Kabat numbering) are described in SEQ ID NOs: 26-31.

To make the anti-TIE2/anti-VEGF bispecific molecule (ITP-221), the VEGF trap sequence from Aflibercept (Eylea® See U.S. Pat. No. 7,279,519) (SEQ ID NO: 39) was attached to the C-terminus of the heavy chain of the anti-TIE2 antibody (VH is SEQ ID NO: 13) having the human IgG4-PAA Fc region (SEQ ID NO: 18) using a linker ((G4S) 2 (SEQ ID NO: 21)+human IgG1 hinge, SEQ ID NO: 22) to produce the full heavy chain of SEQ ID NO: 40. The light chain sequence of ITP-221 is based on that of the 3H7 anti-TIE2 antibody (SEQ ID NO: 25).

To create the anti-TIE2/anti-C5a bispecific molecule (ITP-204), an anti-C5a nanobody sequence (SEQ ID NO: 44, CDRs described in SEQ ID NOs: 41-43) was fused to the C-terminus of the heavy chain of the anti-TIE2 antibody to produce the full heavy chain of SEQ ID NO: 45. This was achieved using a human IgG4-PAA Fc region (SEQ ID NO: 18) with a linker ((G4S) 2 (SEQ ID NO: 21)+human IgG1 hinge, SEQ ID NO: 22). The light chain of ITP-204 is based on that of the 3H7 anti-TIE2 antibody (SEQ ID NO: 25).

### B. Expression and Purification of Fusion Proteins

Plasmid DNAs encoding the multispecific antibodies were transiently co-transfected into ExpiCHO-S™ cells following the manufacturer's instructions (Gibco). Equal amounts of heavy and light chain DNAs were used. The transfected cells were then incubated at 32° C. in a humidified atmosphere of 5% CO₂ in air, with shaking, according to the Max Titer protocol.

After 12 days of incubation, the culture medium containing the secreted fusion protein was collected and centrifuged to remove the cells. The culture supernatant was then filtered. The fusion proteins were purified using the AKTA™ Pure purification system (Cytiva) with a HiTrap™ MabSelect SuRe™ Protein A affinity column (Cytiva). The binding/wash buffer consisted of 20 mM sodium phosphate (pH 7.2) and 150 mM NaCl, while the elution buffer was 25 mM sodium acetate (pH 3.3). The eluted protein fractions were immediately neutralized by adding 100 μl of 1 M Tris-HCl (pH 8.8) per milliliter of fraction.

The pooled fractions were buffer-exchanged into PBS (pH 7.4) using a centrifugal filter unit (Amicon). The final sample was stored until further use. Concurrent quality control studies were conducted on the fusion proteins, including SDS-PAGE analysis (Invitrogen™ Novex™ WedgeWell™), SEC-HPLC (Agilent Infinity 1260 with a 300 Å pore size column), endotoxin measurement (Charles River Cartridge, <0.05 EU/ml sensitivity), and Surface Plasmon Resonance (SPR Biacore™).

### Example 2. Fusion Proteins Bind Both a Target Ligand and Tie2 on the Cell Surface

To investigate whether the bispecific antibodies pre-complexed with a target ligand could bind to human Tie2 (hTie2), a CHO-hTie2 cell line, stably expressing human Tie2 on its surface, was employed in this study along with the commercially available biotinylated target ligand proteins listed in Table 2. The cells were grown to >80% confluence in a 10 cm dish, collected using Accutase® (a cell detachment solution comprised of collagenolytic and proteolytic enzymes), and aliquoted (0.2×10\hat{ }6 cells/tube) into Fluorescent Activated Cell Sorting (FACS) tubes. The cells were washed with 1 mL of FACS buffer and centrifuged for 5 minutes at 200 RCF to pellet the cells. The FACS buffer was aspirated, and the cells were incubated with 200 μL of FACS buffer containing a mixture of bispecific antibodies (10 nM) and the biotinylated TNFα, IL-23, C5a, or VEGF-A (10 nM). After incubating for 1 hour at 4° C., 1 mL of FACS buffer was added to each tube, and the cells were centrifuged and decanted as before. The streptavidin-APC (SA-APC) solution was added to the cells. Samples were incubated for 1 hour at 4° C., washed with FACS buffer, and fixed. Finally, the samples were resuspended in 500 μL of FACS buffer for analysis on an Attune™ N×T flow cytometer.

TABLE 2
Reagents for Biotinylated Target Binding Assay

| | | Cat# or | |
|---|---|---|---|
| Reagent | Company | Project# | Lot# |
| Biotinylated Human | Acro | VE5H82Q025UG | BV5743B- |
| VEGF165, His, | Biosystems | | 2418F1-1K5 |
| Avitag ™ 25 μg | | | |
| Biotinylated Human | BioIntron | 9PYQ5001 | B- |
| C5a Protein, | | | 202404190114 |
| Avitag ™ His Tag | | | |
| Biotinylated Human | Acro | TNAH82E325UG | CBL25cP1- |
| TNF-alpha protein, | Biosystems | | 2267F1-195 |
| His, Avitag ™active | | | |
| trimer, 25 μg | | | |
| Biotinylated human | Acro | ILBH82W625UG | BVG36- |
| IL-23 alpha & IL-12 | Biosystems | | 235GF1- |
| beta heterodimer | | | 1BA |
| protein, His, | | | |
| Avitag ™ and Tag | | | |
| free, 25 μg | | | |
| Allophycocyanin | Biolegend ® | 405207 | B388667 |
| (APC) Streptavidin | | | |
| 100 μg | | | |
| Fetal Bovine Serum | Life | A5670502 | C2930776RP |
| (FBS), Premium, | Technology | | |
| heat-inactivated, | | | |
| One Shot format | | | |
| Dulbecco's | Life | 14190250 | |
| phosphate-buffered | Technology | | |
| saline (DPBS), no | | | |
| calcium, no | | | |
| magnesium | | | |
| 10% Sodium Azide | VWR | 101449-096 | S020920J2201 |
| 100 mL | | | |
| Accutase ® Cell | BD | 561527 | 1232387 |
| Detachment Solution | Biosciences | | |
| 100 mL | | | |

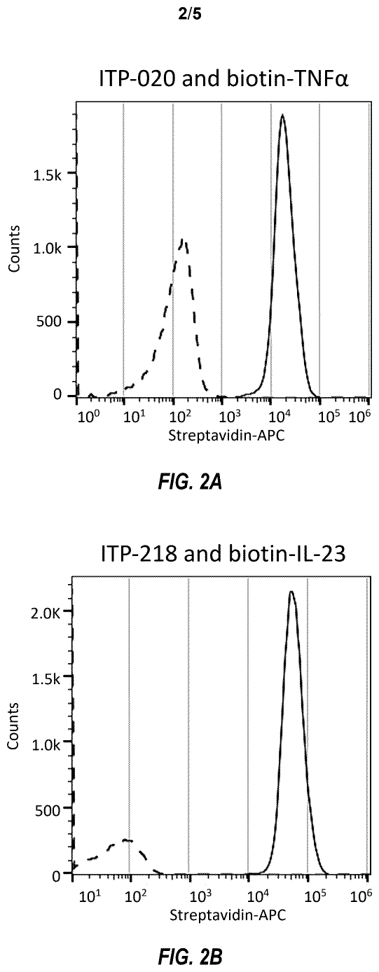

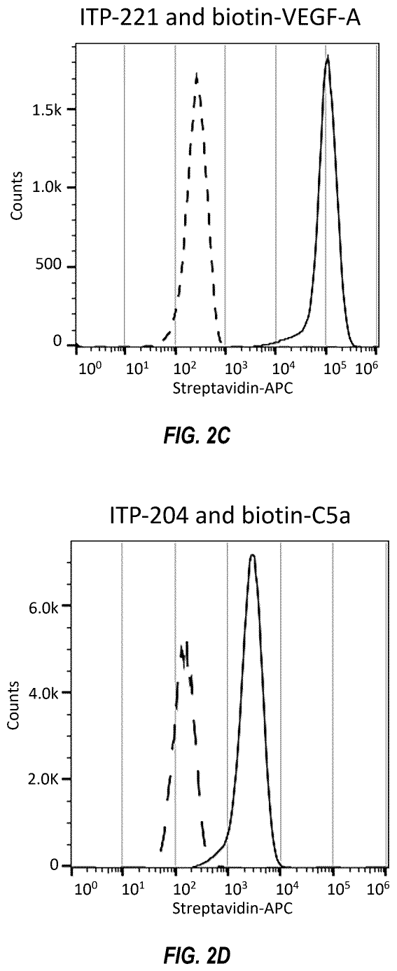

To account for non-specific binding, gating control samples were generated using the cells treated only with the biotinylated cargo (10 nM) during the first incubation or only with SA-APC (1:20) during the second incubation. These control samples were used to establish a true baseline to determine whether the observed binding is non-specific or mediated by the bispecific antibodies. All bispecific antibodies bound to the surface of hTie2-CHO cells and exhibited a higher MFI-APC (mean fluorescence intensity-allophycocyanin) than the control samples (FIGS. 2A-2D).

### Example 3. Internalization of the Target Ligands into Cells

To examine whether the bound target ligand in the tertiary bispecific antibody-target ligand-Tie2 complex is internalized into cells, CHO-hTie2 cells were treated with a bispecific antibody and a biotinylated target for 0 to 16 hours at either 4° C. or 37° C. (See Kim E G et al., Biomolecules 10 (6): 955 (2020)). Incubating the cells at 4° C. prevents any receptor-mediated internalization into cells, thus serving as a baseline. CHO-hTie2 cells were seeded into a 12-well plate (0.5×10\hat{ }6 cells per well) and grown in complete Dulbecco's Modified Eagles's Medium (DMEM, with 10% FBS, 1% pen/strep, 1% non-essential amino acids (NEAA)) until they were more than 70% confluent. The growth media was aspirated, and the cells were washed with Dulbecco's Phosphate Buffered Saline (DPBS). Biotinylated target ligands (see Table 2) at a 10 nM concentration (IL-23, C5a, TNFα, or VEGF-A) in the absence or presence of a bispecific antibody (10 nM) were added to the cells. The cells were then placed at 4° C. for 1 hour to allow the antibodies to bind to hTie2 on the surface of the CHO cells.

After the 1-hour incubation at 4° C., the media was aspirated. The cells were then placed at either 4° C. or 37° C. for the indicated incubation time points (0-16 hours). Following this incubation, the cells were washed twice with FACS buffer containing sodium azide, which acts as a preservative by preventing microbial growth and inhibits further receptor internalization or recycling of receptors. The cells were then stained with 200 μL of FACS buffer containing streptavidin-APC (SA-APC, Biolegend®, 1:20) for 1 hour at 4° C., washed twice with 500 μL of FACS buffer, and collected by treating the cells with Accutase® for 3 minutes followed by gentle trituration. The cells were collected into a 96-well plate, fixed with 2% PFA for 10 minutes, and resuspended in 250 μL of FACS buffer. The samples were analyzed using an Attune™ N×T flow cytometer.

Internalization was determined by calculating the relative mean fluorescence intensity (MFI, % of control)=(MFI_(t=x)/MFI_(t=0))*100. Percent internalization was calculated using the relative MFI as follows (Kim E G et al., Biomolecules June 25;10 (6): 955 (2020)): Internalization (%)=100%-Relative MFI (% of control).

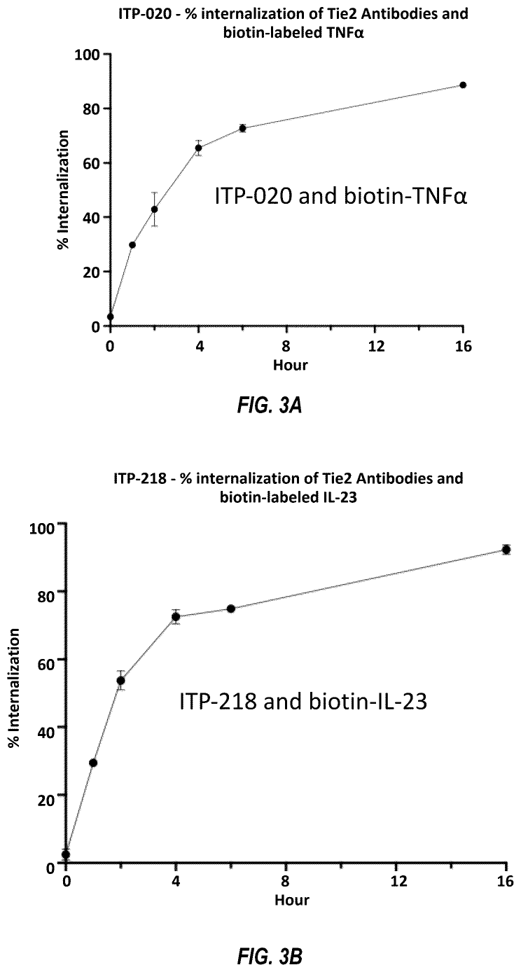

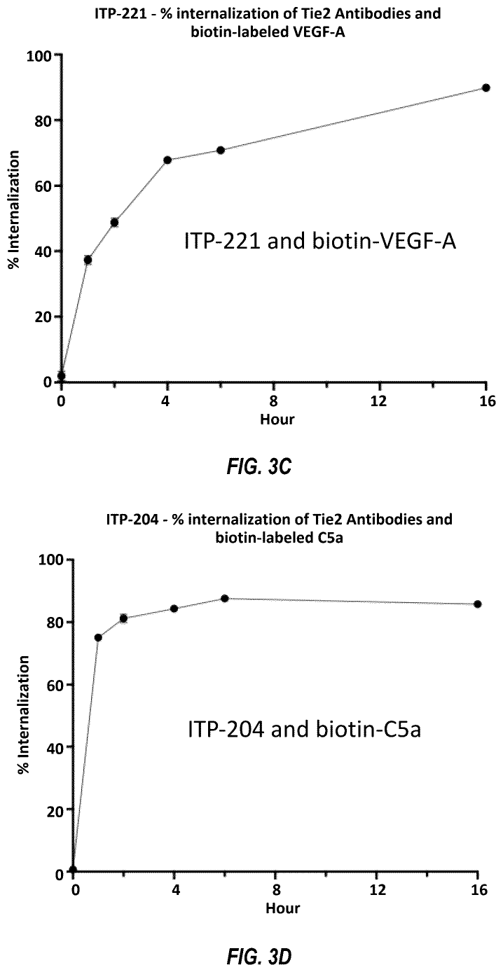

All biotinylated target ligands were internalized (FIGS. 3A-3D). The extent of internalization started low at the earlier time points and increased over time up to 16 hours. These data suggest that internalization of the target ligand occurs with similar kinetics, indicating that the bispecific antibody facilitates the internalization and depletion of a soluble ligand within the cells.

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and should be construed in accordance with the appended claims and any equivalents thereof.

When terms such as “at least” and “about” precede a list of numerical values or ranges, the terms modify all of the values or ranges provided in the list. In some instances, the term about may include numerical values that are rounded to the nearest significant figure.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A fusion protein comprising (a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen-binding domain, wherein the second antigen-binding domain binds to tumor necrosis factor alpha (TNFα), interleukin 23 (IL-23), or complement component 5a (C5a).

2. The fusion protein of claim 1, wherein the anti-Tie2 antibody or antigen-binding fragment thereof comprises a heavy chain variable region comprising heavy chain CDRs comprising the amino acid sequences of SEQ ID NOs: 7-9 and a light chain variable region comprising light chain CDRs comprising the amino acid sequences of SEQ ID NOs: 10⁻¹².

3. The fusion protein of claim 2, wherein the anti-Tie2 antibody or antigen-binding fragment thereof comprises a heavy chain variable region comprising a sequence having at least 80% identity to the amino acid sequence of SEQ ID NO: 13; and/or a light chain variable region comprising a sequence having at least 80% identity to the amino acid sequence of SEQ ID NO: 14.

4. The fusion protein of claim 1, wherein the anti-Tie2 antibody or antigen-binding fragment thereof comprises a heavy chain variable region comprising the amino acid sequence of SEQ ID NO: 13; and/or a light chain variable region comprising the amino acid sequence of SEQ ID NO: 14.

5. (canceled)

6. (canceled)

7. The fusion protein of claim 1, wherein the second antigen-binding domain comprises a TNFα binding domain, and wherein the TNFα binding domain comprises a TNFα receptor domain or an anti-TNFα antibody or antigen-binding fragment thereof.

8. (canceled)

9. The fusion protein of claim 7, wherein the TNFα receptor domain comprises a sequence having at least 80% identity to the amino acid sequence of SEQ ID NO: 23.

10. The fusion protein of claim 9, wherein the TNFα receptor domain comprises the amino acid sequence of SEQ ID NO: 23.

11. (canceled)

12. The fusion protein of claim 7, wherein the anti-TNFα antibody or antigen-binding fragment thereof comprises:
(a) a heavy chain variable region comprising heavy chain CDRs comprising the amino acid sequences of SEQ ID NOs: 47-49 and a light chain variable region comprising light chain CDRs comprising the amino acid sequences of SEQ ID NOs: 50-52;
(b) a heavy chain variable region comprising heavy chain CDRs comprising the amino acid sequences of SEQ ID NOs: 55-57 and a light chain variable region comprising light chain CDRs comprising the amino acid sequences of SEQ ID NOs: 58-60; or
(c) a heavy chain variable region comprising heavy chain CDRs comprising the amino acid sequences of SEQ ID NOs: 63-65 and a light chain variable region comprising light chain CDRs comprising the amino acid sequences of SEQ ID NOs: 66-68.

13. The fusion protein of claim 12, wherein the anti-TNFα antibody or antigen-binding fragment thereof comprises:
(a) a heavy chain variable region comprising an amino acid sequence having at least 80% identity to the amino acid sequence of SEQ ID NO: 53; and/or a light chain variable region comprising an amino acid sequence having at least 80% identity to the amino acid sequence of SEQ ID NO: 54;
(b) a heavy chain variable region comprising an amino acid sequence having at least 80% identity to the amino acid sequence of SEQ ID NO: 61; and/or a light chain variable region comprising an amino acid sequence having at least 80% identity to the amino acid sequence of SEQ ID NO: 62; or
(c) a heavy chain variable region comprising an amino acid sequence having at least 80% identity to the amino acid sequence of SEQ ID NO: 69; and/or a light chain variable region comprising an amino acid sequence having at least 80% identity to the amino acid sequence of SEQ ID NO: 70.

14. The fusion protein of claim 13, wherein the anti-TNFα antibody or antigen-binding fragment thereof comprises:
(a) a heavy chain variable region comprising the amino acid sequence of SEQ ID NO: 53; and/or a light chain variable region comprising the amino acid sequence of SEQ ID NO: 54;
(b) a heavy chain variable region comprising the amino acid sequence of SEQ ID NO: 61; and/or a light chain variable region comprising the amino acid sequence of SEQ ID NO: 62; or
(c) a heavy chain variable region comprising the amino acid sequence of SEQ ID NO: 69; and/or a light chain variable region comprising the amino acid sequence of SEQ ID NO: 70.

15. (canceled)

16. (canceled)

17. (canceled)

18. (canceled)

19. (canceled)

20. (canceled)

21. The fusion protein of claim 1, wherein the second antigen-binding domain comprises an IL-23 binding domain, and wherein the IL-23 binding domain is an anti-IL-23 antibody or antigen-binding fragment thereof.

22. (canceled)

23. The fusion protein of claim 21, wherein the anti-IL-23 antibody or antigen-binding fragment thereof comprises:
(a) a heavy chain variable region comprising heavy chain CDRs comprising the amino acid sequences of SEQ ID NOs: 26-28 and a light chain variable region comprising light chain CDRs comprising the amino acid sequences of SEQ ID NOs: 29-31;
(b) a heavy chain variable region comprising heavy chain CDRs comprising the amino acid sequences of SEQ ID NOs: 71-73 and a light chain variable region comprising light chain CDRs comprising the amino acid sequences of SEQ ID NOs: 74-76;
(c) a heavy chain variable region comprising heavy chain CDRs comprising the amino acid sequences of SEQ ID NOs: 79-81 and a light chain variable region comprising light chain CDRs comprising the amino acid sequences of SEQ ID NOs: 82-84; or
(d) a heavy chain variable region comprising heavy chain CDRs comprising the amino acid sequences of SEQ ID NOs: 87-89 and a light chain variable region comprising light chain CDRs comprising the amino acid sequences of SEQ ID NOs: 90-92.

24. The fusion protein of claim 23, wherein the anti-IL-23 antibody or antigen-binding fragment thereof comprises:
(a) a heavy chain variable region comprising an amino acid sequence having at least 80% identity to the amino acid sequence of SEQ ID NO: 32; and/or a light chain variable region comprising an amino acid sequence having at least 80%, 85%, 95%, 96%, 97%, 98%, or 99% identity to the amino acid sequence of SEQ ID NO: 33;
(b) a heavy chain variable region comprising an amino acid sequence having at least 80% identity to the amino acid sequence of SEQ ID NO: 77; and/or a light chain variable region comprising an amino acid sequence having at least 80% identity to the amino acid sequence of SEQ ID NO: 78;
(c) a heavy chain variable region comprising an amino acid sequence having at least 80% identity to the amino acid sequence of SEQ ID NO: 85; and/or a light chain variable region comprising an amino acid sequence having at least 80% identity to the amino acid sequence of SEQ ID NO: 86; or
(d) a heavy chain variable region comprising an amino acid sequence having at least 80% identity to the amino acid sequence of SEQ ID NO: 93; and/or a light chain variable region comprising an amino acid sequence having at least 80% identity to the amino acid sequence of SEQ ID NO: 94.

25. The fusion protein of claim 24, wherein the anti-IL-23 antibody or antigen-binding fragment thereof comprises:
(a) a heavy chain variable region comprising the amino acid sequence of SEQ ID NO: 32; and/or a light chain variable region comprising the amino acid sequence of SEQ ID NO: 33;
(b) a heavy chain variable region comprising the amino acid sequence of SEQ ID NO: 77; and/or a light chain variable region comprising the amino acid sequence of SEQ ID NO: 78;
(c) a heavy chain variable region comprising the amino acid sequence of SEQ ID NO: 85; and/or a light chain variable region comprising the amino acid sequence of SEQ ID NO: 86; or
(d) a heavy chain variable region comprising the amino acid sequence of SEQ ID NO: 93; and/or a light chain variable region comprising the amino acid sequence of SEQ ID NO: 94.

26. (canceled)

27. (canceled)

28. (canceled)

29. (canceled)

30. (canceled)

31. (canceled)

32. (canceled)

33. (canceled)

34. (canceled)

35. The fusion protein of claim 1, wherein the second antigen-binding domain comprises a C5a binding domain, and wherein the C5a binding domain comprises an anti-C5a nanobody.

36. (canceled)

37. The fusion protein of claim 35, wherein the anti-C5a nanobody comprises a heavy chain variable region comprising heavy chain CDRs comprising the amino acid sequences of SEQ ID NOs: 41-43.

38. The fusion protein of claim 37, wherein the anti-C5a nanobody comprises a sequence having at least 80% identity to the amino acid sequence of SEQ ID NO: 44.

39. The fusion protein of claim 38, wherein the anti-C5a nanobody comprises the amino acid sequence of SEQ ID NO: 44.

40. A fusion protein comprising:
(a) an anti-Tie2 antibody or antigen-binding fragment thereof, wherein the fusion protein binds to Tie2 Ig3-FNIII (1-3) domain, comprising SEQ ID NOs: 2, 3, or 4; and (b) a second antigen binding domain comprising a VEGF-A binding domain, wherein the VEGF-A binding domain comprises a sequence having at least 80% identity to the amino acid sequence of SEQ ID NO: 39; and further wherein the fusion protein has an IgG4 backbone.

41. The fusion protein of claim 40, wherein the VEGF-A binding domain comprises the amino acid sequence of SEQ ID NO: 39.

42. (canceled)

43. (canceled)

44. (canceled)

45. (canceled)

46. (canceled)

47. (canceled)

48. The fusion protein of claim 1, wherein the fusion protein comprises a linker between the second antigen-binding domain and the anti-Tie2 antibody or antigen-binding fragment thereof, wherein the linker: (a) comprises a sequence having between 5 and 50 amino acid residues, between 10 and 40 residues, between 15 and 30 residues, or 20 residues, and/or wherein the linker comprises a sequence having at least 80% identity to the amino acid sequence of SEQ ID NO: 21 or SEQ ID NO: 22.

49. (canceled)

50. The fusion protein of claim 48, wherein the linker comprises the amino acid sequence of SEQ ID NO: 21 or SEQ ID NO: 22.

51. (canceled)

52. (canceled)

53. The fusion protein of claim 1, wherein the anti-Tie2 antigen-binding fragment is an scFv, and wherein the scFv comprises an amino acid sequence having at least 80% identity to the amino acid sequence of SEQ ID NO: 15.

54. (canceled)

55. The fusion protein of claim 53, wherein the anti-Tie2 scFv comprises the amino acid sequence of SEQ ID NO: 15.

56. The fusion protein of claim 1, wherein the fusion protein:
(a) has an IgG isotype;
(b) has an IgG isotype of the IgG1 or IgG4 class;
(c) comprises one or more mutations in a heavy chain constant region that reduce or eliminate interaction with Fc Receptors;
(d) comprises one or more mutations altering L234, L235, G236, and G237 (EU numbering) to a LAGA mutation, a FEGG mutation, an AAGG mutation, an AAGA mutation, a LALA mutation, or a combination thereof; and/or
(e) comprises a CH domain comprising a sequence having at least 80% identity to the amino acid sequence of SEQ ID NO: 16 or SEQ ID NO: 18.

57. (canceled)

58. (canceled)

59. (canceled)

60. (canceled)

61. The fusion protein of claim 56, wherein the fusion protein comprises a CH domain comprising the amino acid sequence of SEQ ID NO: 16 or SEQ ID NO: 18.

62. (canceled)

63. (canceled)

64. (canceled)

65. (canceled)

66. (canceled)

67. (canceled)

68. (canceled)

69. The fusion protein of claim 1, wherein;
(a) the second antigen-binding domain is linked to the C-terminus of the heavy chain (HC) of the anti-Tie2 antibody or antigen-binding fragment thereof, and wherein the fusion protein comprises, C-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof; or
(b) wherein the second antigen-binding domain is linked to the N-terminus of the heavy chain (HC) of the anti-Tie2 antibody or antigen-binding fragment thereof, wherein the fusion protein comprises, N-terminal to the heavy chain constant domain or domains of the anti-Tie2 antibody or antigen-binding fragment thereof and in N- to C-terminal order, a linker between the second antigen-binding domain and the anti-Tie2 antibody or antigen-binding fragment thereof.

70. (canceled)

71. (canceled)

72. (canceled)

73. The fusion protein of claim 1, comprising a heavy chain comprising an amino acid sequence having at least 80% identity to the amino acid sequence of SEQ ID NO: 24; and/or a light chain comprising an amino acid sequence having at least 80% identity to the amino acid sequence of SEQ ID NO: 25.

74. The fusion protein of claim 73, wherein the fusion protein comprises a heavy chain comprising the amino acid sequence of SEQ ID NO: 24; and/or a light chain comprising the amino acid sequence of SEQ ID NO: 25.

75. (canceled)

76. (canceled)

77. The fusion protein of claim 1, comprising: (a) a heavy chain comprising an amino acid sequence having at least 80% identity to the amino acid sequence of any one of SEQ ID NOs: 34-35; and/or a light chain comprising an amino acid sequence having at least 80% identity to the amino acid sequence of SEQ ID NO: 36; or
(b) a heavy chain comprising an amino acid sequence having at least 80% identity to the amino acid sequence of any one of SEQ ID NOs: 40 or 45, and/or a light chain comprising an amino acid sequence having at least 80% identity to the amino acid sequence of SEQ ID NO: 25.

78. The fusion protein of claim 77, wherein the anti-IL-23 antibody comprises:
(a) a heavy chain comprising the amino acid sequence of any one of SEQ ID NOs: 34-35; and/or a light chain comprising the amino acid sequence of SEQ ID NO: 36;
(b) a heavy chain comprising the amino acid sequence of any one of SEQ ID NOs: 40 or 45, and/or a light chain comprising the amino acid sequence of SEQ ID NO: 25.

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. The fusion protein of claim 1, wherein the fusion protein binds to Tie2 IgG3-FNIII (1-3) domain comprising SEQ ID NOs: 2, 3, or 4 with an affinity K_{D} (M) of about 1E-9 to 1E-12 M.

92. A pharmaceutical composition comprising the fusion protein of claim 1 and a pharmaceutically acceptable vehicle, excipient, and/or diluent.

93. A nucleic acid encoding the fusion protein of claim 1.

94. An expression vector comprising the nucleic acid of claim 93.

95. A host cell comprising the nucleic acid of claim 93 or an expression vector comprising the nucleic acid of claim 93.

96. (canceled)

97. (canceled)

98. (canceled)

99. A method of degrading a soluble target ligand comprising administering an effective amount of the fusion protein of claim 1 to the soluble target ligand.

100. (canceled)

101. (canceled)

102. (canceled)

103. A method for reducing the serum concentration of a soluble target ligand in a subject in need thereof, comprising administering an effective amount of the fusion protein of claim 1 to the subject.

104. (canceled)

105. (canceled)

106. A method for preventing or treating an angiogenic disease, comprising administering an effective amount of the fusion protein of claim 1 to a subject in need thereof.

107. (canceled)

108. (canceled)

109. (canceled)

110. (canceled)

111. (canceled)

112. A method for regulating angiogenesis, endothelial signaling, inflammation, and/or vascular leakage, comprising administering an effective amount of the fusion protein of claim 1 to a subject in need thereof.

113. (canceled)

114. (canceled)

115. (canceled)

116. (canceled)
